(19) United States
(12) Patent Application Publication
(10) Pub. No.: US 2007/0253953 A1
(43) Pub. Date: Nov. 1, 2007

(54) Molecular Signature of the Pten Tumor Suppressor

(75) Inventors: Charlie D. Chen, Los Angeles, CA (US); Charles L. Sawyers, New York, NY (US)

Correspondence Address: GATES & COOPER LLP;HOWARD HUGHES CENTER, 6701 CENTER DRIVE WEST, SUITE 1050, LOS ANGELES, CA, 90045, US

(73) Assignee: REGENTS OF THE UNIVERSITY OF CALIFORNIA, THE, Oakland, CA (US)

(21) Appl. No.: 10/582,841

(22) PCT Filed: Dec. 15, 2004
(86) PCT No.: PCT/US04/42258

§ 371 Date: Jul. 9, 2007

Related U.S. Application Data
(60) Provisional application No. 60/530,101, filed on Dec. 15, 2003.

(51) Int. Cl. A61K 39/395; C12Q 1/68

(52) U.S. Cl. 424/138.1; 435/6

(57) ABSTRACT

The present invention relates to the identification a molecular signature for PTEN tumor suppressor. The molecular signature comprising a gene or genes that are of use for diagnosis, prognosis, drug research and development and therapeutics. Specifically, the present invention relates to identification of IGFBP2 gene, its mRNA and/or protein products that closely associate with PTEN mutations. The present invention further demonstrates that IGFBP2 expression is negatively regulated by PTEN, positively regulated by PI3K and Akt activation, that IGFBP2 plays a functional role in the PTEN signaling and is required for Akt transformation. The use of IGFBP2 gene, its gene product such as its RNA transcript, protein and molecular probes in diagnosis, prognosis, drug discovery and validation and therapeutic target and therapeutics is also contemplated.

### BACKGROUND OF THE PRESENT INVENTION

1. Field of Invention

The present invention relates to the identification of genes and their products including their coding protein products that are useful in diagnostics, prognostics and therapeutics of human tumors. In particular, the present invention relates to a set or sets of genes and their products that are associated with tumor suppressor gene PTEN abnormalities, such as PTEN gene deletions, loss of heterozygosity and/or mutations. The present invention relates further to genes and their products that are associated with PTEN-regulated cellular processes, in particular, the PI3K- and/or Akt signal transduction pathways and activations.

2. Description of Related Arts

The PTEN/MMAC1/TEP1 tumor suppressor gene was identified by genomic representational difference analysis (RDA) on human cancer tissues (1), by positional cloning to the genomic locus mutated in multiple advanced cancers (2), and by homologous search for novel protein tyrosine phosphatase (3). This gene is located on human chromosome 10q23, a genomic locus with frequent loss of heterozygosity in multiple advanced cancers. It encodes a protein of 403 amino acids with sequence highly homologous to protein tyrosine phosphatase and the cytoskeletal proteins tensin and auxilin. Germline mutations of PTEN are associated with Cowden and Bannayan-Zonana syndromes, two autosomal dominant disorders characterized as harmartomas with increased susceptibility to cancer (4-6). Somatic mutations of this gene are found in many human cancers, including glioblastoma and prostate cancer with a frequency of up to 50%. Homozygous deletion of the PTEN gene is lethal and heterozygous deletion results in tumor formation in several organs in mice (7-9). These data indicate that PTEN is an important tumor suppressor for a variety of cancers.

One of the well-characterized functions for PTEN is lipid phosphatase activity. This activity dephosphorylates phosphatidylinositol triphosphate at the D3 position, reducing the amount of an important signal transduction molecule produced by PI3K in response to many growth factors, such as insulin-like growth factor 1(IGF-1) that is implicated in tumor formation (10, 11). This PI3K-antagonizing activity in turn inhibits activation of its downstream effector Akt and leads to inhibition of cell survival and proliferation, cellular processes essential for tumor formation and progression (12). In addition to its lipid phosphatase activity, PTEN is also a tyrosine phosphatase that reduces the tyrosine phosphorylation of the focal adhesion kinase (FAK), indicating that PTEN also negatively regulates interactions with the extracellular matrix (13). Furthermore, PTEN deleted mouse fibroblasts have an enhanced cell motility compared to its isogenic wild-type lines. This enhanced cell motility is associated with increased activities of Rac and Cdc42 through its lipid phosphatase activity (14). Taken together, these data indicate that PTEN regulates many cellular processes through a complex map of signal transduction pathways.

Global gene expression analysis is a useful tool to classify and identify tumor types (15, 16), to identify gene involved in tumor formation and progression (17, 18), and predict clinical outcome of cancer patients (19). Recently, it has also been used to identify signatures for tumor metastasis (20). However, its application to identify molecular signatures of a signal transduction pathway involved in tumor formation and progression has not been reported.

### SUMMARY OF THE PRESENT INVENTION

The present invention relates to the identification of genes and their products including their coding protein products that are useful in diagnostics, prognostics and therapeutics of human tumors. In particular, the present invention relates to a set or sets of genes and their products that are associated with tumor suppressor gene PTEN abnormalities, such as PTEN gene deletions, loss of heterozygosity and/or mutations. The present invention relates further to genes and their products that are associated with PTEN-regulated cellular processes, in particular, the PI3K- and/or Akt signal transduction pathways and activations.

The present invention utilizes global gene expression profiling analyses employing gene chip technology to identify transcriptional targets downstream of the complex signal transduction pathways of PTEN. Gene expression profiling was performed on prostate cancer and giloblastoma, two cancer types frequently affected by PTEN mutations. These global gene expression analyses identify a molecular signature that can accurately classify tumor samples according to its PTEN status regardless of tumor types. Extensive studies were carried out for IGFBP2 gene and its protein products, the most significant gene in the signature. It was demonstrated that IGFBP2 is biochemically regulated by PTEN and plays a functional role in PTEN function.

In one embodiment of present invention, a set of genes consists of 490 genes as listed in Table 2 with the Gini index number from highest to the lowest were identified and evaluated for their predictive power of associating with the PTEN status in tumors.

In another embodiment of present invention, a set of genes comprising 12 genes with the highest Gini index were identified and evaluated individually and combined for their predictive power of associating with the PTEN status in tumors.

These genes include insulin-like growth factor binding protein 2 or IGFBP2 (Accession numbers X16302 and S37730), a hypothetical protein (Acc# AF052186), TUA8 Cri-du-chat region (Acc# AF009314), dual specificity phosphatase 10 or MPK-5 (Acc# AB026436), Neuralized (Acc# AF029729), regulator of G-protein signaling 1 or RGS-1 (Acc# S59049), expressed in activated T/LAK lymphocytes or LAP-4p (Acc# AB002405), gamma-tubulin complex protein 2 or GCP2 (Acc# AF042379), human AMP deaminase gene or AMPD3 (Acc# U29926), PFTAIRE protein kinase 1 or PFTK1 (Acc# AB020641), and pleckstrin homology, sec 7 and coiled/coid domains 1 or cytohesin 1 (Acc# M85169).

In yet another embodiment of present invention, individual gene of the above said 12 genes is identified as useful in associating with the PTEN status in tumors, thus, the establishment of diagnostic, prognostic and therapeutic values of these genes and/or their RNA transcripts and/or protein products in human tumors associated with PTEN abnormalities.

In yet another embodiment of present invention, the IGFBP2 gene and its RNA and protein products are identified as closely associated with the PTEN gene abnormalities such as deletions, mutations and loss of heterozygosity.

In a further embodiment of present invention, the IGFBP2 gene and its RNA and protein products are identified to be associated with P13K signal transduction pathway, in particular, PI3K activation and inhibition.

In a further embodiment of present invention, the IGFBP2 gene and its RNA and protein products are identified to be associated with Akt signal transduction pathway, in particular, Akt phosphorylation through activation or inhibition.

In one embodiment of present invention, a diagnostic and/or prognostic product comprising an antibody against the IGFBP2 provides diagnostic and/or prognostic value in associating tumor staging and grading in association with PTEN status.

In a further embodiment of present invention, the IGFBP2 gene and its RNA and protein products are useful in the screening and selection of therapeutic useful drugs against human cancers.

In another embodiment of present invention, a diagnostic and/or prognostic product comprising an antibody against the IGFBP2 is useful in screening and selecting a therapeutic drug for treating human cancers.

In further embodiment of present invention, the IGFBP2 gene, its product such as RNA transcript and/or protein product are useful in designing, screening, validating and developing a therapeutically useful drug or means such as a dominant negative IGFBP2 that is useful in abolishing IGFBP2 normal and/or abnormal functions in promoting cancer formation, progression, antisense RNA, antisense oligonucleotide and/or siRNAi compounds, or shRNA gene knockdown technology that suppress or erase IGFBP2 gene expression or reduce its RNA transcript level; antibodies that neutralize IGFBP2 functionalities, gene therapies that embody the IGFBP2 gene.

In another embodiment of present invention, a diagnostic and/or prognostic product comprising an antibody against the IGFBP2 provides diagnostic and/or prognostic value in predicting the effectiveness and/or responsiveness of a therapeutics for treating human cancers.

In one embodiment of present invention, a diagnostic and/or prognostic product comprising a molecular probe in the forms of a nucleic acid molecule such as oligonucleotide, DNA and/or RNA molecule with its nucleotide sequence homologous or complementary to the gene sequence of the IGFBP2 gene provides diagnostic and/or prognostic value in associating tumor staging and grading in association with PTEN status.

In one embodiment of present invention, a diagnostic and/or prognostic product comprising a molecular probe in the forms of a nucleic acid molecule such as oligonucleotide, DNA and/or RNA molecule with its nucleotide sequence homologous or complementary to the gene sequence of the IGFBP2 gene is useful in the screening and selection of therapeutic useful drugs against human cancers.

In one embodiment of present invention, a diagnostic and/or prognostic product comprising a molecular probe in the forms of a nucleic acid molecule such as oligonucleotide, DNA and/or RNA molecule with its nucleotide sequence homologous or complementary to the gene sequence of the IGFBP2 gene provides diagnostic and/or prognostic value in predicting the effectiveness and responsiveness of a therapeutics for human cancers.

In another embodiment of present invention, a diagnostic and/prognostic product comprising a gene probe or an antibody against its product is useful in diagnosis and/or prognosis of human cancers, in selecting and screening a therapeutic compounds of means for treating human cancers and/or in predicting effectiveness and responsiveness of a therapeutic means including a therapeutic drug in the treatment of human cancers. The above said gene is selected from a group of 490 genes enlisted in the Table 2, in particular, is selected from a group of genes consisting the followings 12 genes: insulin-like growth factor binding protein 2 or IGFBP2 (accession numbers X16302 and S37730), a hypothetical protein (ACC# AF052186), TUA8 CRI-DU-CHAT region (ACC#AF009314), dual specificity phosphatase 10 or MPK-5 (ACC# AB026436), neuralized (ACC# AF029729), Regulator of G-Protein Signaling 1 or RGS-1 (ACC#S59049), expressed in activated T/LAK lymphocytes or LAP-4P (ACC# AB002405), gamma-tubulin complex protein 2 or GCP2 (ACC# AF042379), human amp dearninase gene or AMPD3 (ACC# U29926), pftaire protein kinase 1 or PFTK1 (ACC# AB020641), and pleckstrin homology, SEC 7 and coiled/coid domains 1 or cythohesin 1 (ACC# M85169).

The present invention relates with utilizing the above identified genes in Table 2, in particular the above said 12 genes, especially the IGFBP2 gene and its products as drug target in screening and selecting therapeutically useful compounds and/or means for the treatment of human cancers.

### BRIEF DESCRIPTION OF THE DRAWINGS

FIG. 1. Molecular signature of the PTEN tumor suppressor. A. Predictive power of each gene represented by Gini index. B. Ability of sets of genes to predict PTEN status. C. 12 genes separate tumors according to the PTEN status. D. Hierachical clustering of genes against tumors.

FIG. 2. Upregulation of IGFBP-2 in PTEN mutated tumors. (A, B, C) Western blot analysis of prostate cancer xenograft samples (A), glioblastoma tissue samples (B and C). D and E. Radioassay for hIGFBP2 in culture media (D) and serum of mice carrying xenograft tumors (E).

FIG. 3. IGFBP2 is regulated by the PTEN/Akt pathway. Western blot analysis of the PTEN-mutated or wild type mouse embryonic fibroblasts (A), LNCaP cells treated with vehicle or PI3K inhibitor (13), LAPC4 cells with or without overexpression of constitutive-active Akt (C).

FIG. 4. IGFBP2 rescued growth inhibition by PTEN. Acutely infected PC3 cells with viruses carrying different cDNAs were subject to cell count (A) or cell cycle analysis (B), and the IGFBP2 expression was determined by western blot analysis (C).

FIG. 5. IGFBP2 plays a functional role in the PI3K-Akt pathway. (A) Cell cycle analysis of vector or IGFBP2 infected LNCaP cells treated with PI3K inhibitor (LY294002). (B and C) Clonagenic assay on wild-type or IGFBP2 knockout mouse embryonic fibroblasts with or without constitutive-active Akt expression (B), or myc expression (C).

FIG. 6. IGFBP2 knockdown decreased the growth of PTEN mutated prostate cancer cells, an effect identical to re-introduction of exogenous wild-type PTEN. Top panel, cell count on vector, shRNA targeting IGFBP2, or PTEN infected PC3 cells; bottom panel, western blot analysis on the engineered cells. p FIG. 7. (A) Overexpression of AR is the cause of hormone refractory prostate cancer. (B) Hormone refractory prostate cancer is still ligand dependent refractory prostate cancer and can be used as a screening method for prostate cancer drug development.

### DETAILED DESCRIPTION OF THE PREFERRED EMBODIMENT

To identify transcriptional targets downstream of the complex signal transduction pathways of PTEN, we performed a gene expression profiling on prostate cancer and glioblastoma, two cancer types frequently affected by PTEN mutations. This global gene expression analysis identifies a molecular signature that can accurately classify tumor samples according to its PTEN status regardless of tumor types. We also studied IGFBP2, the most significant gene in the signature. We demonstrated that IGFBP2 is biochemically regulated by PTEN and plays a functional role in PTEN function.

### A. Definitions

To facilitate understanding of the invention, a number of terms are defined below:

Nucleotide: a monomeric unit of DNA or RNA consisting of a sugar moiety (pentose), a phosphate, and a nitrogenous heterocyclic base. The base is linked to the sugar moiety via the glycosidic carbon (1′ carbon of the pentose) and that combination of base and sugar is a nucleoside. A nucleoside containing at least one phosphate group bonded to the 3′ or 5′ position of the pentose is a nucleotide.

Base Pair (bp): a partnership of adenine (A) with thymine (T), or of cytosine (C) with guanine (G) in a double stranded DNA molecule. In RNA, uracil (U) is substituted for thymine. Generally the partnership is achieved through hydrogen bonding.

Nucleic Acid: a polymer of nucleotides, either single or double stranded.

Gene: a nucleic acid whose nucleotide sequence codes for an RNA or a polypeptide. A gene can be either RNA or DNA.

cDNA: a single stranded DNA that is homologous to an MrRNA sequence and does not contain any intronic sequences.

Sense: a nucleic acid molecule in the same sequence order and composition as the homolog mRNA. The sense conformation is indicated with a “+”, “s” or “sense” symbol.

Antisense: a nucleic acid molecule complementary to the respective mRNA molecule. The antisense conformation is indicated as a “−” symbol or with a “a” or “antisense” in front of the DNA or RNA, e.g., “aDNA” or “aRNA”.

Template: a nucleic acid molecule being copied by a nucleic acid polymerase. A template can be single-stranded, double-stranded or partially double-stranded, depending on the polymerase. The synthesized copy is complementary to the template, or to at least one strand of a double-stranded or partially double-stranded template. Both RNA and DNA are synthesized in the 5′ to 3′ direction. The two strands of a nucleic acid duplex are always aligned so that the 5′ ends of the two strands are at opposite ends of the duplex (and, by necessity, so then are the 3′ ends).

Nucleic Acid Template: a double-stranded DNA molecule, double stranded RNA molecule, hybrid molecules such as DNA-RNA or RNA-DNA hybrid, or single-stranded DNA or RNA molecule.

Oligonucleotide: a molecule comprised of two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof

Primer: an oligonucleotide complementary to a template. The primer complexes with the template to yield a primer/template duplex for initiation of synthesis by a DNA polymerase. The primer/template complex is extended during DNA synthesis by the addition of covalently bonded bases linked at the 3′ end, which are complementary to the template. The result is a primer extension product. Virtually all known DNA polymerases (including reverse transcriptases) require complexing of an oligonucleotide to a single-stranded template (“priming”) to initiate DNA synthesis. A primer is selected to be “substantially” or “sufficiently” complementary to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence has sufficient complementarity with the sequence of the template to hybridize and thereby form a template/primer complex for synthesis of the extension product of the primer.

Complementary or Complementarity or Complementation: used in reference to polynucleotides (i.e., a sequence of nucleotides) related by the base-pairing rules. For example, the sequence “A-G-T” is complementary to the sequence “T-C-A,” and also to “T-C-U.” Complementation can be between two DNA strands, a DNA and an RNA strand, or between two RNA strands. Complementarity may be “partial” or “complete” or “total”. Partial complementarity or complementation occurs when only some of the nucleic acid bases are matched according to the base pairing rules. Complete or total complementarity or complementation occurs when the bases are completely matched between the nucleic acid strands. The degree of complementarity between nucleic acid strands has significant effects on the efficiency and strength of hybridization between nucleic acid strands. This is of particular importance in amplification reactions, as well as in detection methods that depend on binding between nucleic acids. Percent complementarity or complementation refers to the number of mismatch bases over the total bases in one strand of the nucleic acid. Thus, a 50% complementation means that half of the bases were mismatched and half were matched. Two strands of nucleic acid can be complementary even though the two strands differ in the number of bases. In this situation, the complementation occurs between the portion of the longer strand corresponding to the bases on that strand that pair with the bases on the shorter strand.

Homologous or homology: refers to a polynucleotide sequence having similarities with a gene or mRNA sequence. A nucleic acid sequence may be partially or completely homologous to a particular gene or mRNA sequence, for example. Homology may also be expressed as a percentage determined by the number of similar nucleotides over the total number of nucleotides.

Complementary Bases: nucleotides that normally pair up when DNA or RNA adopts a double stranded configuration.

Complementary Nucleotide Sequence: a sequence of nucleotides in a single-stranded molecule of DNA or RNA that is sufficiently complementary to that on another single strand to specifically hybridize between the two strands with consequent hydrogen bonding.

Conserved: a nucleotide sequence is conserved with respect to a preselected (reference) sequence if it non-randomly hybridizes to an exact or total complement of the preselected sequence.

Hybridize and Hybridization: the formation of complexes between nucleotide sequences which are sufficiently complementary to form complexes via complementary base pairing. Where a primer (or splice template) “hybridizes” with target (template), such complexes (or hybrids) are sufficiently stable to serve the priming function required by a DNA polymerase to initiate DNA synthesis. There is a specific, i.e. non-random, interaction between two complementary polynucleotide that can be competitively inhibited.

Nucleotide Analog: a purine or pyrimidine nucleotide that differs structurally from T. G. C, or U, but is sufficiently similar to substitute for the normal nucleotide in a nucleic acid molecule.

DNA Homolog: a nucleic acid having a preselected conserved nucleotide sequence and a sequence coding for a receptor capable of binding a preselected ligand.

Amplification: nucleic acid replication involving template specificity. Template specificity is frequently described in terms of “target” specificity. Target sequences are “targets” in that they are sought to be sorted out from other nucleic acids. Amplification techniques have been designed primarily for this sorting. Template specificity is achieved in most amplification techniques by the choice of enzyme.

Enzymatic Amplification: a method for increasing the concentration of a segment in a target sequence from a mixture of nucleic acids without cloning or purification.

Polymerase Chain Reaction (PCR): an amplification reaction is typically carried out by cycling i.e., simultaneously performing in one admixture, the first and second primer extension reactions, each cycle comprising polynucleotide synthesis followed by denaturation of the double stranded polynucleotides formed. Methods and systems for amplifying a DNA homolog are described in U.S. Pat. Nos. 4,683,195 and 4,683,202, both to Mullis et al.

Amplifiable Nucleic Acid and Amplified Products: nucleic acids that may be amplified by any amplification method.

DNA-dependent DNA Polymerase: an enzyme that synthesizes a complementary DNA copy from a DNA template. Examples are DNA polymerase 1 from E. coli and bacteriophage T7 DNA polymerase. Under suitable conditions a DNA-dependent DNA polymerase may synthesize a complementary DNA copy from an RNA template.

DNA-dependent RNA Polymerase or Transcriptase: enzymes that synthesize multiple RNA copies from a double stranded or partially double stranded DNA molecule having a promoter sequence. Examples of transcriptases include, but are not limited to, DNA-dependent RNA polymerase from E. coli and bacteriophage T7, T3, and SP6.

RNA-dependent DNA Polymerase or Reverse Transcriptase: enzymes that synthesize a complementary DNA copy from an RNA template. All known reverse transcriptases also have the ability to make a complementary DNA copy from a DNA template. Thus, reverse transcriptases are both RNA-dependent and DNA-dependent DNA polymerases.

RNase H: an enzyme that degrades the RNA portion of an RNA/DNA duplex. RNase H may be an endonuclease or an exonuclease. Most reverse transcriptase enzymes normally contain an RNase H activity. However, other sources of RNase H are available, without an associated polymerase activity. The degradation may result in separation of the RNA from a RNA/DNA complex. Alternatively, the RNase H may simply cut the RNA at various locations such that pieces of the RNA melt off or are susceptible to enzymes that unwind portions of the RNA.

Reverse Transcription: the synthesis of a DNA molecule from an RNA molecule using an enzymatic reaction in vitro. For example, the RNA molecule may be primed with a primer that is complementary to the RNA molecule and the DNA molecule is synthesized by extension using a reverse transcriptase such as the DNA polymerase with reverse transcription activity, MMLV reverse transcriptase, AMV reverse transcriptase, and any other enzyme that has the ability to synthesize a DNA molecule from an RNA molecule template.

In Vitro Transcription: the synthesis of an RNA molecule from a DNA molecule using an enzymatic reaction in vitro. For example, the DNA molecule may be double stranded and comprises an RNA polymerase promoter such as T7, SP6, T3, or any other enzyme. promoter for synthesis of RNA from DNA.

Vector: a recombinant nucleic acid molecule such as recombinant DNA (rDNA) capable of movement and residence in different genetic environments. Generally, another nucleic acid is operatively linked therein. The vector can be capable of autonomous replication in a cell in which case the vector and the attached segment is replicated. One type of preferred vector is an episome, i.e., a nucleic acid molecule capable of extrachromosomal replication. Preferred vectors are those capable of autonomous replication and/or expression of nucleic acids to which they are linked. Vectors capable of directing the expression of genes encoding for one or more polypeptides are referred to herein as “expression vectors”. Particularly important vectors allow cloning of cDNA from mRNAs produced using a reverse transcriptase.

Functional parts: a portion of an intact molecule that retains one or more desired properties of the intact molecules. Thus, for example, an antibody binds an antigen. In that context of the property of binding that antigen, a functional part of an antibody can be any portion of an antibody that binds the cognate antigen. Similarly, a functional part of a nucleic acid that encodes an antibody that binds that antigen is any portion of that nucleic acid that encodes a polypeptide that binds to that antigen.

Antibody: in various grammatical forms as used herein refers to immunoglobulin molecules and immunologically active portions of immunoglobulin molecules, i.e., molecules that contain a combining site for antigen or paratope. Exemplary antibody molecules are intact immunoglobulin molecules, substantially intact immunoglobulin molecules and portions of an immunoglobulin molecules, including those portions known in the art as F_{ab}, F_{ab's} (F_{ab′})₂, Fᵥ and scFᵥ.

Immunoreact: in various forms means specific binding between an antigenic determinant-containing molecule and a molecule containing an antibody combining site such as a whole antibody molecule or a portion thereof.

Cistron: a sequence of nucleotides in a DNA molecule coding for an amino acid residue sequence and including upstream and downstream DNA expression control elements.

Promoter: a nucleic acid to which a polymerase molecule recognizes, perhaps binds to, and initiates synthesis. For the purposes of the instant invention, a promoter can be a known polymerase binding site, an enhancer and the like, any sequence that can initiate synthesis by a desired polymerase.

Knockdown: a method to which a RNA made from a DNA sequence (shRNA) introduced into a cell or a RNA sequence (siRNA) introduced into a cell to initiate degradation of the mRNA of a protein of interest.

### B. Methods

The present invention provides a novel method for identifying and selecting genes that associate with PTEN gene abnormalities and/or PTEN-related cellular process, and/or PI3K- and/or Akt-related signal transduction pathway. The present invention combines the following elements as discussed in details thereafter:

1. isolation of nucleic acids (genomic DNAs or mRNAs) from tumor cells, tumor specimens;

2.preparation of tumor samples for probing microarrays or gene chips;

3.performing gene chip hybridization with tumor samples;

4.Random Forest (RF) computational analyses of the gene chip datasets;

5. identifying genes with predictive power for association with PTEN status tumor and cancer cells.

The invention now will be exemplified further in the following non-limiting examples.

### EXAMPLES

### Example 1

### Random Forest Deals Effectively with Microarray Data

We have used microarray technology to identify overexpression of androgen receptor as the general mechanism for hormone refractory prostate cancer. The data indicate that overexpression of androgen receptor is a diagnostic and therapeutic target for hormone refractory prostate cancer and can be used as a screening method for hormone refractory prostate cancer drug development (FIG. 7). This is consistent with microarray technology being a useful tool for a variety of purposes. However, it has been difficult in identifying molecular signatures for signal transduction pathways. One of the reasons is that microarray experiments are usually performed on a relatively few samples, therefore, data analysis on these experiments requires specific statistical tools. In this report, we described a novel unsupervised learning algorithm, called Random Forest, to identify a molecular signature for the signaling pathway of PTEN. This statistic tool can deal effectively with small data sets involving relatively a few observations (samples) and a large volume of variables (gene expression values). It can calculate a predictive power for each gene. When a set of genes is used to predict the PTEN status, it can also generate an error rate by a three-fold cross-validation, in which one-third of the samples are left out as test set Therefore, identification and verification of signatures, and identification of significant genes can be achieved using this algorithm.

### Example 2

### Molecular Signature of the PTEN Tumor Suppressor

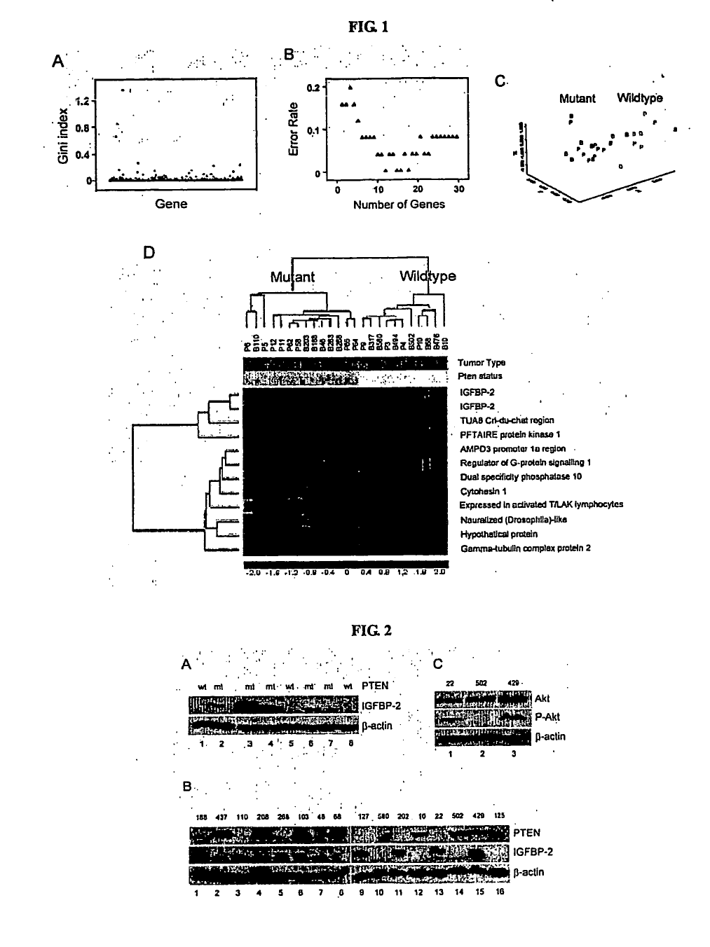

To identify transcriptional targets associate with the PTEN tumor suppressor function, we compared the gene expression profiles of 11 tissue samples that have the wild-type PTEN gene to those of 14 samples that have mutated PTEN gene (table 1). These 25 samples include 12 advanced prostate cancer xenografts and 13 glioblastoma tissue samples. The PTEN status of the prostate cancer xenografts were characterized previously (21) and those of the glioblastoma were determined by western blot and genomic DNA sequence analysis. Six of the glioblastoma samples do not express the PTEN protein and, therefore, were defined as PTEN mutant samples. The other seven samples have the wild-type PTEN because they express the PTEN protein and they do not carry point mutation, which was determined by genomic DNA sequence analysis. We used both prostate cancer and glioblastoma tissue samples in order to increase a tissue-independent signature associated with the PTEN status. We adopted a statistic technique called Random Forest (RF) because this method has been designed to analyze data that contain many covariates and relatively few observations (Breiman L, 1999). This technique is ideal to analyze microarray data, in which expression of a large number of genes is observed in a relatively few samples. This approach identified 490 genes that have statistic power in predicting the PTEN status, and ranked each gene according to the significance of its predictive power, which is represented by Gini index (FIG. 1A).

We next ask how many genes must be included in order to correctly predict the PTEN status, since most of the 490 genes have weak predictive power (their Gini indexes are near zero). We generated an error rate for each gene set by a three-fold cross-validation, in which one-third of the samples were left out as test sets. The first gene in the gene set was the one with the most predictive power (the highest Gini index), and a following gene was added each time according to its ranking of the Gini index. A gene list cannot predict the PTEN status accurately until 12 genes with the highest Gini indexes were included (FIG. 1B). The accurate prediction was maintained in gene lists composed of the “top” 18 genes and was lost when more genes were included, consistent with the fact that each gene has different Gini index and, therefore, carries different weights in predicting the PTEN status. The accuracy of the 12 genes to predict the PTEN status in the test set was confirmed by multidimensional scaling analysis (FIG. 1C) and by hierarchical clustering (FIG. 1D). Glioblastoma and prostate cancer were clustered into PTEN mutant or wild type tumor regardless of cancer types. Further studies are required to determine whether this signature can be exploited more broadly as a tool to define PTEN status of tumors using larger, independent datasets with characterized PTEN status tumors.

### Example 3

### Elevated Levels of IGFBP2 Expression in PTEN Mutant Tumors

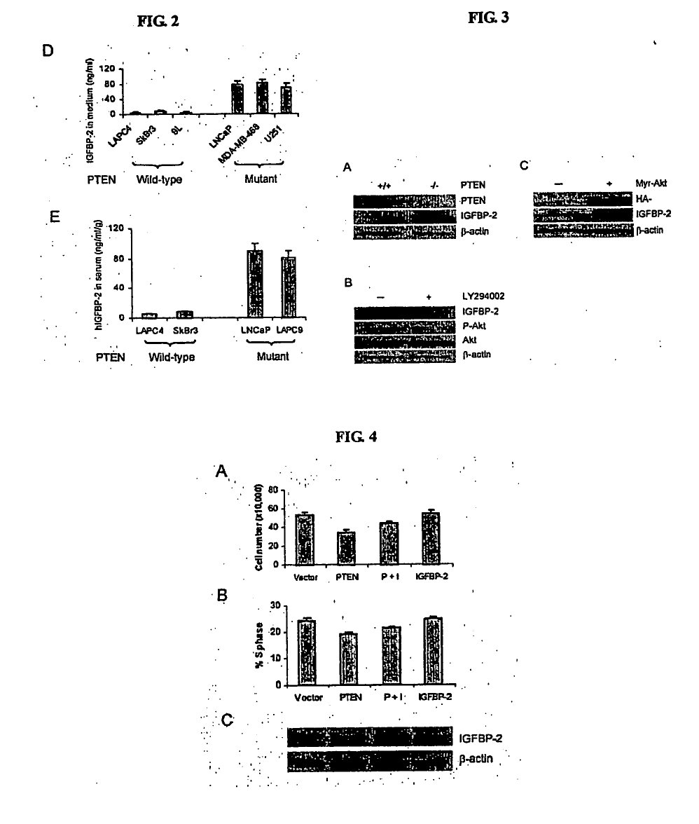

Having identified several genes whose expression patterns correlate with the PTEN status, we wish to investigate biochemical regulation and biological role of one of these genes in PTEN function. Because both probe sets in the microarray were identified and both have the highest power in predicting the PTEN status, IGFBP2 was chosen for further study. As the first step, we confirmed the relationship between PTEN mutations and IGFBP2 expression by western blot analysis using whole tissue lysates from prostate cancer xenografts. IGFBP2 protein was detected in PTEN mutated xenografts LAPC9, LUCaP 35, and LNCaP but not in PTEN wild-type tumors LAPC4 and LUCaP23 (FIG. 2A, table 1), consistent with the microarray analysis.

The relationship was extended to other tumors that were not included in the microarray analysis. IGFBP2 was highly expressed in PTEN mutated tumors LAPC3, LAPC12, and LUCaP41, but was not detected in PTEN wild-type tumor LAPC14 (FIG. 2A, table 1). This association also holds true for 23 of the 24 glioblastoma samples examined, of which 13 samples were included in the microarray analysis and 10 of them were independent samples (FIG. 2B, table 1). IGFBP2 protein was detected in samples whose PTEN expression was low or lost, but was not detected in samples whose PTEN expression was high. Genomic sequence analysis indicate that the PTEN protein detected in the western blot analysis was wild-type. The correlation was confirmed by immunohistochemical analysis (unpublished data, Paul Mischel).

There is one exception for the association between the PTEN mutations and IGFBP2 expression in glioblastoma samples (FIG. 2B, table 1). This sample (#429) has PTEN protein expression while IGFBP2 is also highly expressed. Genomic sequence analysis indicates that this sample has the wild-type PTEN gene, suggesting that mechanisms other than PTEN mutations are responsible. Indeed, this sample has high levels of Akt and Akt activation, as indicated by western blot analysis on total Akt and phosphorylation of ser 473 of Akt (FIG. 2C). The mechanism of elevated Akt level in this specific patient is unknown.

To examine if high level of IGFBP2 is secreted by cells with PTEN mutations, glioblastoma (9L and U251) and prostate cancer cells (LAPC4 and LNCaP) were grown in tissue culture and the IGFBP2 levels were measured by radioimmunoassay. In contrast to less than 20 ng/ml of secreted IGFBP2 by cells with wild-type PTEN genes, the levels of secreted protein in cells with mutated PTEN gene are more than 70 ng/ml (FIG. 2D). The high levels of secreted IGFBP2 were also detected in PTEN mutated breast cancer cell (MDA-MB-468), but not in PTEN wild-type breast cancer cell (SkBr3).

To examine if serum IGFBP2 levels correlate with PTEN status in tumors, serum levels of human IGFBP2 were measured from mice carrying human prostate and breast cancer xenografts (FIG. 2E). While serum levels of human IGFBP2 levels were low in mice carrying human tumors with the wild-type PTEN genes, mice with PTEN mutated tumors contained high levels of human IGFBP2 in serum. These data indicate that human tumors with PTEN mutations secreted high levels of IGFBP2, and raise the possibility that serum IGFBP-2 levels could serve as a biomarker for PTEN status.

### Example 4

### Inhibition of IGFBP2 Expression by PTEN

To establish a causal role of PTEN loss in IGFBP-2 upregulation, we extended our analysis to an isogenic model. Western blot analysis was performed using the lysates from the PTEN wild-type and deleted isogenic mouse embryonic fibroblasts (MEF) (22). While IGFBP2 protein was barely detectable in PTEN wild-type MEF, PTEN mutant cells produced a high level of IGFBP2 (FIG. 3A).

To determine if upregulation of the IGFBP2 expression by the PTEN mutations is dependent upon the PI3K/Akt pathway, pharmacological and genetic approaches were employed. When PTEN mutated cells were treated with a pharmacological drug (LY294002) that inhibits the PI3K kinase activity, the production of IGFBP2 was reduced to the basal level (FIG. 3B). Furthermore, IGFBP2 was induced in cells with the wild-type PTEN gene when a constitutively active Akt allele was expressed (FIG. 3C). These results indicate that IGFBP2 expression is induced by the PI3K/Akt pathway, which is antagonized by the PTEN tumor suppressor.

### Example 5

### Functional Role of IGFBP2 in the PTEN/Akt Signaling

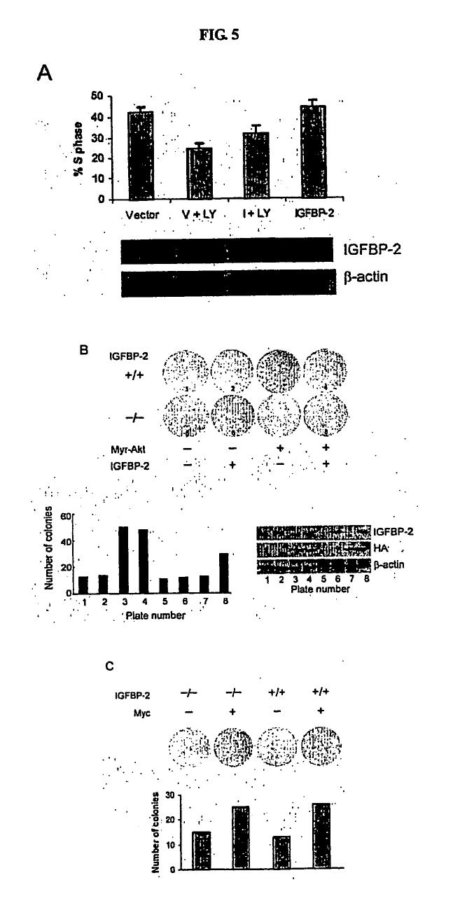

To determine if IGFBP2 plays a functional role in the PTEN signal transduction pathway, we introduced either PTEN or IGFBP-2 into PTEN null cell lines by lentiviral infection, which gives highly efficient infection rates in prostate cancer epithelial cells (>90%). As reported, re-introduction of the wild-type PTEN decreased growth of PC3 cells by 36% (FIG. 4A), with a concomitant decrease of the endogenous IGFBP2 expression (FIG. 4C). Forced expression of exogenous IGFBP2 rescued the growth inhibition of PTEN by 47% (FIG. 4A). A similar effect was observed with cell cycle analysis (FIG. 4B). Re-introduction of the wild-type PTEN into PC3 cell reduced the percentage of cells in S phase, and the effect is partially rescued by forced expression of exogenous IGFBP2. These data suggest that down-regulation of IGFBP2 may partially contribute to the PTEN tumor suppressor function.

To examine if IGFBP2 is involved in the PI3K signaling, a pharmacological approach was employed. Consistent with the result in FIG. 3B, LNCaP cells have a high basal level of Akt activation. Treatment of LY294002, a specific PI3K inhibitor, resulted in reduced Akt phosphorylation and IGFBP2 expression (FIG. 5A, bottom panel). This treatment caused a decrease of the percentage of cells in S phase by 40%, and the reduction was partially (28%) rescued by the forced expression of exogenous IGFBP2 (FIG. 5A, top panel). To examine if IGFBP2 plays a biological role for Akt function, clonogenic assay was performed using IGFBP2 knockout MEF (23). While Akt promoted colony formation in IGFBP2 wild type MEF (FIG. 5B, top and left panel), deletion of IGFBP2 abrogated this promoting activity. The requirement of IGFBP2 is specific to Akt because c-myc promoted colony formation in both cells (FIG. 5C). The inability of Akt to promote colony formation in IGFBP2 knockout MEF can be rescued by re-introduction of IGFBP2 (FIG. 5B). Taken together, these results indicate that one of the effects of the PTEN tumor suppressor is to suppress the expression of IGFBP2, which is involved in the function of the PI3K/Akt signal transduction pathway.

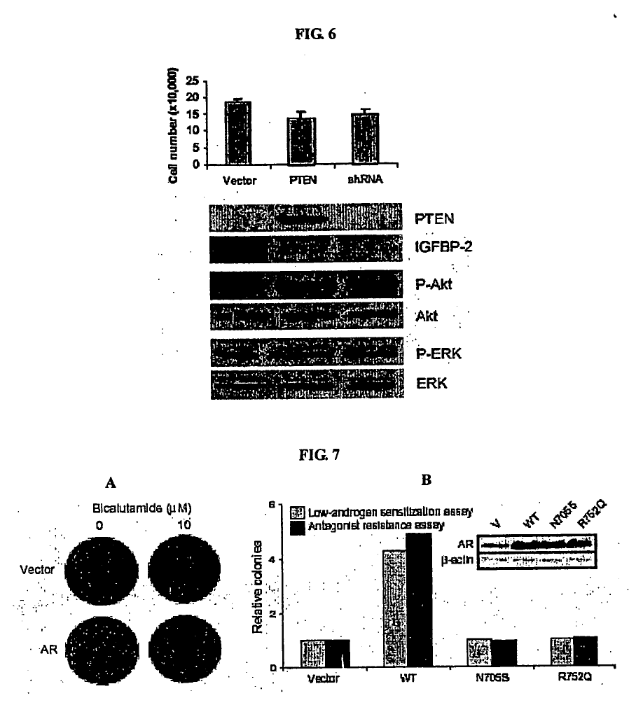

To determine how IGFBP2 is involved in Akt function, we made use of gene knockdown technology by shRNA. The shRNA efficiently knockdown IGFBP2 expression, as shown by western blot analysis (FIG. 6). This knockdown reduced the growth of PC3 and the activation of Akt, effects identical to re-introduction of the wild-type PTEN (FIG. 6). These data suggest that IGFBP2 may regulate Akt activation through an autoloop mechanism.

### Example 6

### IGFBP2 is a Surrogate Marker for PTEN

Through random forest and other statistical analysis, we identified upregulation of IGFBP2 expression as the most consistent change associated with PTEN mutations. Among 12559 probe sets in the microarray, both probe sets representing IGFBP2 were identified as the most and the second most significant gene to predict the PTEN status. We demonstrated that IGFBP2 is biochemically regulated by PTEN and PI3K-Akt pathway. Consistent with our finding, it was reported that overexpression of IGFBP2 was only observed in glioblastoma, but not in low- or intermediate-grade gliomas (24). In addition, IGFBP2 overexpression was observed in 50% of glioblastoma. The stage in which IGFBP2 is overexpressed and the percentage of tumors with this gene overexpression coincide with the frequency of PTEN mutations in advanced gliomas (25). Overexpression of IGFBP2 was also identified as the most distinct progression-related expression change in high-grade gliomas in another similar study through cDNA microarrays and tissue arrays (26). This study uncovered that IGFBP2 is a poor prognostic marker for patients with gliomas. While patients with IGFBP2 negative tumors had a mean survival of 75 months, patients with tumors of strong IGFBP2 expression had a mean survival of 23 months. This also coincides with the aggressiveness of PTEN mutated tumors. These data suggest that upregulation of IGFBP2 in PTEN mutated tumors may play an important role in tumor formation and progression. Indeed, forced expression of IGFBP2 partially rescued the inhibitory effect of PTEN and a PI3K inhibitor as well (FIG. 4).

### Example 7

### Serum IGFBP2 can be Developed as a Surrogate Marker for PTEN Mutations and Akt Activation

We and other have recently demonstrated that tumors with PTEN mutations are more sensitive to drugs such as CCI-779 that targets mTOR, a downstream effector of the PI3K/Akt pathway (22, 27). This effect is later observed in several other studies. These studies suggest that drugs targeting the PI3K/Alkt pathway may only benefit patients who have aberrant PTEN/Akt activities. Since PTEN mutations are carried in less than 50% of tumors even for the most frequently mutated cancer type, the pharmaceutical benefit can be masked by an unselected population. This may explain why CCI-779 and some other drugs targeting the PI3K-Akt pathway fail in clinical trials, even though this drug effectively inhibits PTEN mutated cancer cells. Because IGFBP2 is a serum protein, we envision that the serum level of IGFBP2 can be used to predict PTEN mutations and Akt activation. In support of this notion, we detected high concentrations of human IGFBP2 in condition medium of PTEN mutated cells and also in sera of mice carrying PTEN mutated human tumors. In addition, serum concentration of IGFBP2 was shown to be elevated in 50% of patients with advanced prostate cancer (personal communication, Pinchas Cohen). The stage in which IGFBP2 is overexpressed and the percentage of tumors with this gene overexpression coincide with the frequency of PTEN mutations in advanced prostate cancer in patients. Furthermore, it was reported that patients treated with IGF-l, a stimulus for Akt activation, caused an elevated level of IGFBP2 in serum (28). Serum level of IGFBP2 can also be used to predict if drugs hit targets because overexpression of IGFBP2 can be inhibited by a PI3K inhibitor.

### Example 8

### Potential Downstream Targets of PTEN

The smallest gene expression signature associated with the PTEN status contained eight down-regulated and four up-regulated genes in PTEN mutated tumors (FIG. 1D). Several of the identified genes were involved in different pathways implicated in tumor formation and progression. Human neuralized belongs to a family of the neurogenic genes and is an E3 ligase for the Notch signal transduction pathway that is associated with tumorigenesis (29, 30). This protein mediates proteosome-dependent degradation of the Notch ligand Delta (31). Loss-of-function mutations of the neurogenic genes produce hyperplasia of the embryonic nervous system (32), which is reminiscent of phenotype of the brain-specific PTEN knockout mice (33). Furthermore, expression of human neuralized is high in normal human brain tissue, but low or absent in advanced gliomas (34), consistent with our finding. These data suggest that the notch pathway may play an important role in PTEN tumor suppressor function. Dual specificity phosphatase 10, also called MKP-5, selectively dephosphorylates JNK and reduces its activity (35). The level of this phosphatase is reduced in tumors with PTEN loss, suggesting that upregulation of the JNK signal transduction pathway is a key element for cancer development and progression in PTEN-null tumors. This hypothesis is supported by our unpublished data. Curiously, two proteins identified in the signature specifically bind PIP3 (36, 37), the established substrate for the PTEN tumor suppressor. Cytohesin-l belongs to a family of guanine nucleotide-exchange proteins for the 20-kDa ADP ribosylation factor (ARF) (38). It also associates with integrin beta2 and regulate cell adhesion that is important for tumorigenesis and cancer metastasis (39). Regulator of G-protein signaling 1 belongs to a family of GTPase-activating protein and is inhibited by PIP3 (37). These data suggest that a feedback control may be invoked to maintain the PI3K signaling, consistent with a published report that expression of PTEN causes feedback upregulation of IRS-2 (40). Our data suggest that these molecules, particularly these 12 molecules identified through microarray analysis, can be diagnostic and therapeutic targets for PTEN mutated tumors. Current efforts are directed to understand the involvement of these molecules in PTEN tumor suppressor function.

### References

All references cited herein and herein incorporated by reference in entirety. Breiman, L. Random forests-random features. Technical Report 567, Department of Statistics, University of California, Berkeley, September 1999.

1. J. Li et al, Science 275, 1943-7. (1997).

2. P. A. Steck et al., Nat Genet 15, 356-62. (1997).

3. D. M. Li, H. Sun, Proc Natl Acad Sci USA 95, 15406-11. (1998).

4. L. Simpson, R. Parsons, Exp Cell Res 264, 2941. (2001).

5. J. Paez, W. R. Sellers, Cancer Treat Res 115, 145-67 (2003).

6. M. L. Sulis, R. Parsons, Trends Cell Biol 13, 478-83 (September 2003).

7. V. Stambolic et al., Cancer Res 60, 3605-11 (Jul. 1, 2000).

8. K. Podsypanina et al., Proc Natl Acad Sci USA 96, 1563-8 (Feb. 16, 1999).

9. A. Di Cristofano, B. Pesce, C. Cordon-Cardo, P. P. Pandolfi, Nat Genet 19, 348-55 (August 1998).

10. J. M. Chan et al., Science 279, 563-6 (Jan. 23, 1998).

11. S. J. Moschos, C. S. Mantzoros, Oncology 63, 317-32 (2002).

12. I. Vivanco, C. L. Sawyers, Nat Rev Cancer 2, 489-501 (July 2002).

13. M. Tamura et al., Science 280, 1614-7 (Jun. 5, 1998).

14. J. Liliental et al., Curr Biol 10, 4014 (Apr. 6, 2000).

15. T. R. Golub et al., Science 286, 531-7 (Oct. 15, 1999).

16. C. M. Perou et al., Nature 406, 747-52 (Aug. 17, 2000).

17. E. A. Clark T. R. Golub, E. S. Lander, R. O. Hynes, Nature 406, 532-5 (Aug. 3, 2000).

18. S. Varambally et al., Nature 419, 624-9 (Oct. 10, 2002).

19. D. Singh et al., Cancer Cell 1, 203-9 (March 2002).

20. S. Ramaswamy, K. N. Ross, E. S. Lander, T. R. Golub, Nat Genet 33, 49-54 (January 2003).

21. Y. E. Whang et a., Proc Natl Acad Sci USA 95, 5246-50 (Apr. 28, 1998).

22. M. S. Neshat et al., Proc Natl Acad Sci USA 98, 10314-9 (Aug. 28, 2001).

23. T. L. Wood, L. E. Rogler, M. E. Czick, A. G. Schuller, J. E. Pintar, Mol Endocrinol 14, 1472-82 (September 2000).

24. G. N. Fuller et al., Cancer Res 59, 4228-32. (1999).

25. P. L. Dahia, Endocr Relat Cancer 7, 115-29 (June 2000).

26. S. L. Sallinen et al., Cancer Res 60, 6617-22. (2000).

27. K. Podsypanina et al., Proc Natl Acad Sci USA 98, 10320-5 (Aug. 28,2001).

28. R. V. Bhat, T. M. Engber, Y. Zhu, M. S. Miller, P. C. Contreras, J Pharmacol Exp Ther 281, 522-30. (1997).

29. 1. Maillard, W. S. Pear, Cancer Cell 3, 203-5 (March 2003).

30. S. Weijzen et al., Nat Med 8, 979-86 (September 2002).

31. E. C. Lai, Curr Biol 12, R74-8 (Jan. 22, 2002).

32. G. L. Boulianne, A. de la Concha, J. A. Camnpos-Ortega, L. Y. Jan, Y. N. Jan, Embo J 10, 2975-83 (October 1991).

33. M. Groszer et al., Science 294, 2186-9 (Dec 7, 2001).

34. H. Nakamura et al., Oncogene 16, 1009-19 (Feb. 26, 1998).

35. A. Theodosiou, A. Smith, C. Gillieron, S. Arkinstall, A. Ashworth, Oncogene 18, 6981-8 (Nov. 25, 1999).

36. J. K. Klarlund et al., Science 275, 1927-30 (Mar. 28, 1997).

37. S. G. Popov, U. M. Krishna, J. R. Falck, T. M. Wilkie, J Biol Chem 275, 18962-8 (Jun. 23, 2000).

38. J.Cherfils et al., Nature392, 101-5 (Mar. 5, 1998).

39. W. Kolanus et al., Cell 86, 23342 (Jul. 26, 1996).

40. L. Simpson et al., Mol Cell Biol 21, 3947-58 (June 2001).

This application is related to U.S. Pat. No. 10/701,490, filed Nov. 5, 2003, the entire contents of which are incorporated herein by reference. Throughout this application, various publications are referenced. The disclosures of these publications are hereby incorporated by reference herein in their entireties.

TABLE 1
Supplementary Table 1. Validation of the inversed relationship between IGFBP-2
expression and the PTEN mutation in tumors
Samples in microarray analysis

| IGFBP2 | | | | IGFBP2 | | | |
|---|---|---|---|---|---|---|---|
| expression | | | | expression | | | |
| Prostate | RNA (1) | Protein (2) | PTEN status | GBM | RNA | Protein | PTEN status |
| 3 | 354 | no | Wildtype | 10 | 14942 | no | Wildtype |
| 4 | 1807 | no | Wildtype | 68 | 655 | no | Wildtype |
| 9 | 1402 | no | Wildtype | 317 | 3749 | no | Wildtype |
| 10 | 1131 | no | Wildtype | 476 | 16483 | no | Wildtype |
| 5 | 3364 | + | Mutant | 494 | 10699 | no | Wildtype |
| 6 | 10861 | + | Mutant | 502 | 19414 | low | Wildtype |
| 11 | 7770 | +++ | Mutant | 580 | 9131 | no | Wildtype |
| 12 | 11237 | +++ | Mutant | 46 | 37265 | + | Mutant |
| 58 | 11063 | ++ | Mutant | 110 | 22298 | +++ | Mutant |
| 62 | 17217 | ++ | Mutant | 188 | 48838 | +++ | Mutant |
| 64 | 8378 | ++ | Mutant | 203 | 38755 | + | Mutant |
| 65 | 4695 | ++ | Mutant | 263 | 35151 | ++ | Mutant |
| | | | | 268 | 42889 | +++ | Mutant |

Samples not included in microarray analysis

| | |
|---|---|
| IGFBP2 expression | IGFBP2 expression |

| Prostate | Protein | PTEN status | GBM | Protein | PTEN status |
|---|---|---|---|---|---|
| LAPC14 | no | Wildtype | 64 | no | Wildtype |
| LAPC3 | +++++ | Mutant | 103 | no | Wildtype |
| LAPC12 | ++++ | Mutant | 125 | no | Wildtype |
| LuCaP41 | ++ | Mutant | 155 | no | Wildtype |
| | | | 208 | no | Wildtype |
| | | | 305 | no | Wildtype |
| | | | 429 | +++ | Wildtype |
| | | | 437 | no | Wildtype |
| | | | 22 | +++++ | Mutant |
| | | | 127 | ++ | Mutant |
| | | | 202 | +++ | Mutant |

TABLE 2
Supplementary Table 2. A list of 490 genes with statistic powers in predicting the PTEN status

| Rank | ProbeSet | Gene name | Gini Index |
|---|---|---|---|
| 1 | 40422_at | insulin-like growth factor binding protein 2 (36 kD) | 1.39 |
| 2 | 1741_s_at | “S37730/FEATURE = cds/DEFINITION = S37712S4 insulin-like growth factor binding protein- | 0.87 |
| | | 2 [human, placenta, Genomic, 1342 nt, segment 4 of 4]” | |
| 3 | 40026_g_at | hypothetical protein | 0.68 |
| 4 | 36061_at | Cluster Incl. AF009314: Homo sapiens clone TUA8 Cri-du-chat region mRNA/ | 0.28 |
| | | cds = UNKNOWN/gb = AF009314/gi = 2331117/ug = Hs.49476/len = 1463 | |
| 5 | 38555_at | dual specificity phosphatase 10 | 0.24 |
| 6 | 32717_at | neuralized (Drosophila)-like | 0.23 |
| 7 | 36575_at | regulator of G-protein signalling 1 | 0.17 |
| 8 | 32116_at | expressed in activated T/LAK lymphocytes | 0.16 |
| 9 | 39918_at | gamma-tubulin complex protein 2 | 0.15 |
| 10 | 38463_s_at | “Cluster Incl. U29926: Human AMP deaminase (AMPD3) gene, promoter 1a region/ | 0.14 |
| | | cds = (453,2777)/gb = U29926/gi = 1002661/ug = Hs.83918/len = 4018” | |
| 11 | 36502_at | PFTAIRE protein kinase 1 | 0.14 |
| 12 | 38666_at | “pleckstrin homology, Sec7 and coiled/coil domains 1(cytohesin 1)” | 0.12 |
| 13 | 37055_at | ets variant gene 1 | 0.1 |
| 14 | 38812_at | “laminin, beta 2 (laminin S)” | 0.1 |
| 15 | 35414_s_at | jagged 1 (Alagille syndrome) | 0.1 |
| 16 | 33807_at | phosphoinositol 3-phosphate-binding protein-2 | 0.1 |
| 17 | 38415_at | “protein tyrosine phosphatase type IVA, member 2” | 0.09 |
| 18 | 34993_at | “sarcoglycan, delta (35 kD dystrophin-associated glycoprotein)” | 0.08 |
| 19 | 40971_at | KIAA0229 protein | 0.07 |
| 20 | 1398_g_at | mitogen-activated protein kinase kinase kinase 11 | 0.07 |
| 21 | 36935_at | RAS p21 protein activator (GTPase activating protein) 1 | 0.07 |
| 22 | 885_g_at | “integrin, alpha 3 (antigen CD49C, alpha 3 subunit of VLA-3 receptor)” | 0.07 |
| 23 | 35275_at | “adaptor-related protein complex 1, gamma 1 subunit” | 0.06 |
| 24 | 40866_at | “NIPSNAP, C. elegans, homolog 1” | 0.06 |
| 25 | 1506_at | “interleukin 2 receptor, gamma (severe combined immunodeficiency)” | 0.06 |
| 26 | 1910_s_at | B-cell CLL/lymphoma 2 | 0.06 |
| 27 | 36212_at | Cluster Incl. AL049218: Homo sapiens mRNA; cDNA DKFZp564I1916 (from clone | 0.06 |
| | | DKFZp564I1916)/cds = UNKNOWN/gb = AL049218/gi = 4499947/ug = Hs.234793/len = 1474 | |
| 28 | 31530_at | acetyl-Coenzyme A carboxylase beta | 0.06 |
| 29 | 37276_at | IQ motif containing GTPase activating protein 2 | 0.05 |
| 30 | 41028_at | ryanodine receptor 3 | 0.05 |
| 31 | 38336_at | KIAA1013 protein | 0.05 |
| 32 | 1060_g_at | “neurotrophic tyrosine kinase, receptor, type 3” | 0.05 |
| 33 | 37432_g_at | Protein inhibitor of activated STAT X | 0.05 |
| 34 | 34348_at | “serine protease inhibitor, Kunitz type, 2” | 0.05 |
| 35 | 884_at | “integrin, alpha 3 (antigen CD49C, alpha 3 subunit of VLA-3 receptor)” | 0.05 |
| 36 | 33453_at | “ATPase, H+ transporting, lysosomal (vacuolar proton pump), subunit 1” | 0.05 |
| 37 | 36659_at | “collagen, type IV, alpha 2” | 0.05 |
| 38 | 38110_at | syndecan binding protein (syntenin) | 0.05 |
| 39 | 41385_at | erythrocyte membrane protein band 4.1-like 3 | 0.05 |
| 40 | 41176_at | hypothetical protein FLJ12443 | 0.05 |
| 41 | 32174_at | “solute carrier family 9 (sodium/hydrogen exchanger), isoform 3 regulatory factor 1” | 0.05 |
| 42 | 32571_at | “methionine adenosyltransferase II, alpha” | 0.05 |
| 43 | 40935_at | hypothetical protein MGC11308 | 0.04 |
| 44 | 39391_at | associated molecule with the SH3 domain of STAM | 0.04 |
| 45 | 232_at | “M55210/FEATURE = mRNA#1/DEFINITION = HUMLB2A26 Human laminin B2 chain gene, | 0.04 |
| | | exon 28” | |
| 46 | 38650_at | Cluster Incl. L27560: Human insulin-like growth factor binding protein 5 (IGFBP5) mRNA/ | 0.04 |
| | | cds = UNKNOWN/gb = L27560/gi = 452059/ug = Hs.103391/len = 3658 | |
| 47 | 34508_r_at | KIAA1079 protein | 0.04 |
| 48 | 41789_r_at | KIAA0669 gene product | 0.04 |
| 49 | 41132_r_at | heterogeneous nuclear ribonucleoprotein H2 (H‘) | 0.04 |
| 50 | 40210_at | “RAB13, member RAS oncogene family” | 0.03 |
| 51 | 1293_s_at | glycosylphosphatidylinositol specific phospholipase D1 | 0.03 |
| 52 | 39174_at | nuclear receptor coactivator 4 | 0.03 |
| 53 | 37640_at | hypoxanthine phosphoribosyltransferase 1 (Lesch-Nyhan syndrome) | 0.03 |
| 54 | 1287_at | ADP-ribosyltransferase (NAD+; poly (ADP-ribose) polymerase) | 0.03 |
| 55 | 39082_at | annexin A6 | 0.03 |
| 56 | 35247_at | “small nuclear RNA activating complex, polypeptide 5, 19 kD” | 0.03 |
| 57 | 39797_at | KIAA0349 protein | 0.03 |
| 58 | 39550_at | chromosome 1 open reading frame 17 | 0.03 |
| 59 | 33993_at | “myosin, light polypeptide 6, alkali, smooth muscle and non-muscle” | 0.03 |
| 60 | 38030_at | KIAA0332 protein | 0.03 |
| 61 | 410_s_at | “casein kinase 2, beta polypeptide” | 0.03 |
| 62 | 153_f_at | “H2B histone family, member R” | 0.03 |
| 63 | 32695_at | bombesin-like receptor 3 | 0.03 |
| 64 | 33050_at | dopamine receptor D5 | 0.03 |
| 65 | 40399_r_at | mesenchyme homeo box 2 (growth arrest-specific homeo box) | 0.03 |
| 66 | 1434_at | phosphatase and tensin homolog (mutated in multiple advanced cancers 1) | 0.03 |
| 67 | 41035_at | KIAA0775 gene product | 0.03 |
| 68 | 1535_at | checkpoint suppressor 1 | 0.03 |
| 69 | 32769_at | KIAA0993 protein | 0.02 |
| 70 | 33155_at | “Cluster Incl. M95740: Human alpha-L-iduronidase gene/cds = (0.1961)/gb = M95740/ | 0.02 |
| | | gi = 178412/ug = Hs.89560/len = 2234” | |
| 71 | 38769_at | a disintegrin and metalloproteinase domain 12 (meltrin alpha) | 0.02 |
| 72 | 491_at | “U46116/FEATURE = mRNA/DEFINITION = HSPTPRG28 Human receptor tyrosine | 0.02 |
| | | phosphatase gamma (PTPRG) gene, exon 30 and complete cds” | |
| 73 | 34397_at | acid-inducible phosphoprotein | 0.02 |
| 74 | 41131_f_at | heterogeneous nuclear ribonucleoprotein H2 (H‘) | 0.02 |
| 75 | 31995_g_at | brefeldin A-inhibited guanine nucleotide-exchange protein 2 | 0.02 |
| 76 | 1346_at | metallothionein 3 (growth inhibitory factor (neurotrophic)) | 0.02 |
| 77 | 36672_at | prolylcarboxypeptidase (angiotensinase C) | 0.02 |
| 78 | 36188_at | general transcription factor IIIA | 0.02 |
| 79 | 31773_at | cytochrome b-561 | 0.02 |
| 80 | 40504_at | paraoxonase 2 | 0.02 |
| 81 | 41277_at | “sin3-associated polypeptide, 18 kD” | 0.02 |
| 82 | 35239_at | emerin (Emery-Dreifuss muscular dystrophy) | 0.02 |
| 83 | 32774_at | “NADH dehydrogenase (ubiquinone) 1 beta subcomplex, 8 (19 kD, ASHI)” | 0.02 |
| 84 | 39993_at | “phosphatidylinositol glycan, class A (paroxysmal nocturnal hemoglobinuria)” | 0.02 |
| 85 | 41693_r_at | carnitine O-octanoyltransferase | 0.02 |
| 86 | 41866_s_at | “dystrobrevin, beta” | 0.02 |
| 87 | 38607_at | transmembrane 4 superfamily member 5 | 0.02 |
| 88 | 41431_at | MAK-related kinase | 0.02 |
| 89 | 38264_at | RAB interacting factor | 0.02 |
| 90 | 1396_at | L27560/FEATURE = mRNA/DEFINITION = HUMIGFBP5X Human insulin-like growth factor | 0.02 |
| | | binding protein 5 (IGFBP5) mRNA | |
| 91 | 40792_s_at | triple functional domain (PTPRF interacting) | 0.02 |
| 92 | 37511_at | B9 protein | 0.02 |
| 93 | 41814_at | “fucosidase, alpha-L-1, tissue” | 0.02 |
| 94 | 37998_at | superkiller viralicidic activity 2 (S. cerevisiae homolog)-like | 0.02 |
| 95 | 32059_at | Cluster Incl. U79282: Human clone 23801 mRNA sequence/cds = UNKNOWN/gb = U79282/ | 0.02 |
| | | gi = 1710254/ug = Hs.155572/len = 1694 | |
| 96 | 39134_at | target of myb1 (chicken) homolog | 0.02 |
| 97 | 35148_at | “amyloid beta (A4) precursor protein-binding, family A, member 3 (X11-like 2)” | 0.02 |
| 98 | 1804_at | “kallikrein 3, (prostate specific antigen)” | 0.02 |
| 99 | 719_g_at | “protease, serine, 11 (IGF binding)” | 0.02 |
| 100 | 32642_at | chondroitin sulfate proteoglycan 3 (neurocan) | 0.02 |
| 101 | 38096_f_at | “major histocompatibility complex, class II, DP beta 1” | 0.02 |
| 102 | 41124_r_at | ectonucleotide pyrophosphatase/phosphodiesterase 2 (autotaxin) | 0.02 |
| 103 | 36444_s_at | “small inducible cytokine subfamily A (Cys-Cys), member 16” | 0.02 |
| 104 | 41256_at | eukaryotic translation elongation factor 1 delta (guanine nucleotide exchange protein) | 0.02 |
| 105 | 31418_at | high-mobility group (nonhistone chromosomal) protein 17-like 1 | 0.02 |
| 106 | 40679_at | “solute carrier family 6 (neurotransmitter transporter, betaine/GABA), member 12” | 0.02 |
| 107 | 172_at | “inositol polyphosphate-5-phosphatase, 145 kD” | 0.02 |
| 108 | 41308_at | C-terminal binding protein 1 | 0.02 |
| 109 | 41238_s_at | “Cluster Incl. M18700: Human elastase III A gene/cds = (18,827)/gb = M18700/gi = 806625/ | 0.02 |
| | | ug = Hs.181289/len = 918” | |
| 110 | 32264_at | granzyme M (lymphocyte met-ase 1) | 0.02 |
| 111 | 1779_s_at | pim-1 oncogene | 0.02 |
| 112 | 39778_at | “mannosyl (alpha-1,3-)-glycoprotein beta-1,2-N-acetylglucosaminyltransferase” | 0.02 |
| 113 | 1579_at | “M27288/FEATURE = cds/DEFINITION = HUMOCS3 Human oncostatin M gene, exon 3” | 0.02 |
| 114 | 38043_at | 2.19 gene | 0.02 |
| 115 | 33712_at | “sulfortranferase family 4A, member 1” | 0.02 |
| 116 | 32918_at | Cluster Incl. AL080182: Homo sapiens mRNA; cDNA DKFZp434O151 (from clone | 0.02 |
| | | DKFZp434O151)/cds = UNKNOWN/gb = AL080182/gi = 5262658/ug = Hs.225129/len = 1454 | |
| 117 | 35155_at | “casein kinase 1, gamma 2” | 0.02 |
| 118 | 1977_s_at | v-ets avian erythroblastosis virus E26 oncogene homolog 1 | 0.02 |
| 119 | 34147_g_at | 8-oxoguanine DNA glycosylase | 0.02 |
| 120 | 41525_at | high-mobility group 20B | 0.02 |
| 121 | 32068_at | complement component 3a receptor 1 | 0.02 |
| 122 | 37331_g_at | “aldehyde dehydrogenase 4 family, member A1” | 0.02 |
| 123 | 39017_at | Lsm1 protein | 0.02 |
| 124 | 34435_at | aquaporin 9 | 0.02 |
| 125 | 35939_s_at | “POU domain, class 4, transcription factor 1” | 0.02 |
| 126 | 34226_at | mitogen-activated protein kinase kinase kinase kinase 5 | 0.02 |
| 127 | 39602_at | DKFZP586F1018 protein | 0.02 |
| 128 | 33576_at | KIAA0918 protein | 0.02 |
| 129 | 1569_r_at | “L42243/FEATURE = exon#3/DEFINITION = HUMIFNAM08 Homo sapiens (clone 51H8) | 0.02 |
| | | alternatively spliced interferon receptor (IFNAR2) gene, exon 9 and complete cds s” | |
| 130 | 34916_s_at | “tumor necrosis factor receptor superfamily, member 4” | 0.02 |
| 131 | 38696_at | CGG triplet repeat binding protein 1 | 0.02 |
| 132 | 37964_at | ring finger protein 3 | 0.01 |
| 133 | 32598_at | nel (chicken)-like 2 | 0.01 |
| 134 | 36603_at | “GCN1 (general control of amino-acid synthesis 1, yeast)-like 1” | 0.01 |
| 135 | 37737_at | protein-L-isoaspartate (D-aspartate) O-methyltransferase | 0.01 |
| 136 | 36097_at | immediate early protein | 0.01 |
| 137 | 36781_at | “serine (or cysteine) proteinase inhibitor, clade A (alpha-1 antiproteinase, antitrypsin), member 1” | 0.01 |
| 138 | 36919_r_at | formyl peptide receptor 1 | 0.01 |
| 139 | 32963_s_at | Rag D protein | 0.01 |
| 140 | 41409_at | basement membrane-induced gene | 0.01 |
| 141 | 36529_at | hypothetical protein MGC2650 | 0.01 |
| 142 | 41526_at | high-mobility group 20B | 0.01 |
| 143 | 40004_at | sine oculis homeobox (Drosophila) homolog 1 | 0.01 |
| 144 | 41858_at | FGF receptor activating protein 1 | 0.01 |
| 145 | 40270_at | cell division cycle 2-like 5 (cholinesterase-related cell division controller) | 0.01 |
| 146 | 38606_at | “tryptophan 2,3-dioxygenase” | 0.01 |
| 147 | 41758_at | chromosome 22 open reading frame 5 | 0.01 |
| 148 | 36093_at | KIAA0614 protein | 0.01 |
| 149 | 33161_at | “integrin, beta 1 (fibronectin receptor, beta polypeptide, antigen CD29 includes MDF2, | 0.01 |
| | | MSK12)” | |
| 150 | 1028_at | “U43431/FEATURE=/DEFINITION = HSU43431 Human DNA topoisomerase III mRNA, | 0.01 |
| | | complete cds” | |
| 151 | 1557_at | p21/Cdc42/Rac1-activated kinase 1 (yeast Ste20-related) | 0.01 |
| 152 | 32736_at | HSPC022 protein | 0.01 |
| 153 | 34654_at | myotubularin related protein 1 | 0.01 |
| 154 | 39768_at | “Cluster Incl. D13146: Homo sapiens gene for 2,3-cyclic-nucleotide 3-phosphodiesterase/ | 0.01 |
| | | cds = (90,1355)/gb = D13146/gi = 219399/ug = Hs.150741/len = 2594” | |
| 155 | 40424_at | proline synthetase co-transcribed (bacterial homolog) | 0.01 |
| 156 | 524_at | postmeiotic segregation increased (S. cerevisiae) 1 | 0.01 |
| 157 | 36125_s_at | RNA-binding protein (autoantigenic) | 0.01 |
| 158 | 857_at | “protein phosphatase 1A (formerly 2C), magnesium-dependent, alpha isoform” | 0.01 |
| 159 | 36833_at | Bruton agammaglobulinemia tyrosine kinase | 0.01 |
| 160 | 40464_g_at | karyopherin (importin) beta 2 | 0.01 |
| 161 | 39965_at | “ras-related C3 botulinum toxin substrate 3 (rho family, small GTP binding protein Rac3)” | 0.01 |
| 162 | 38458_at | “Cluster Incl. L39945: Human cytochrome b5 (CYB5) gene/cds = (120,548)/gb = L39945/ | 0.01 |
| | | gi = 703082/ug = Hs.83834/len = 836” | |
| 163 | 37748_at | KIAA0232 gene product | 0.01 |
| 164 | 40454_at | FAT tumor suppressor (Drosophila) homolog | 0.01 |
| 165 | 34740_at | forkhead box O3A | 0.01 |
| 166 | 36011_at | syntaxin 10 | 0.01 |
| 167 | 37568_at | Cluster Incl. U79242: Human clone 23560 mRNA sequence/cds = UNKNOWN/gb = U79242/ | 0.01 |
| | | gi = 1710189/ug = Hs.79981/len = 1614 | |
| 168 | 40229_at | target of myb1 (chicken) homolog-like 1 | 0.01 |
| 169 | 37838_at | coagulation factor XII (Hageman factor) | 0.01 |
| 170 | 1128_s_at | chemokine (C—C motif) receptor 1 | 0.01 |
| 171 | 33798_at | KIAA0732 protein | 0.01 |
| 172 | 31728_at | “major histocompatibility complex, class II, DN alpha” | 0.01 |
| 173 | 38947_at | “protein phosphatase, EF hand calcium-binding domain 1” | 0.01 |
| 174 | 1993_s_at | “breast cancer 1, early onset” | 0.01 |
| 175 | 35951_at | neurexin 3 | 0.01 |
| 176 | 35642_at | metaxin 2 | 0.01 |
| 177 | 32061_at | hypothetical protein FLJ10871 | 0.01 |
| 178 | 40195_at | “H2A histone family, member X” | 0.01 |
| 179 | 121_at | paired box gene 8 | 0.01 |
| 180 | 40446_at | divalent cation tolerant protein CUTA | 0.01 |
| 181 | 38642_at | activated leucocyte cell adhesion molecule | 0.01 |
| 182 | 33293_at | lifeguard | 0.01 |
| 183 | 35575_f_at | zinc finger protein 253 | 0.01 |
| 184 | 38595_r_at | KIAA0284 protein | 0.01 |
| 185 | 833_at | “U40279/FEATURE = cds/DEFINITION = HSITGAD06 Human beta-2 integrin alphaD subunit | 0.01 |
| | | (ITGAD) gene, exons 25-30, and partial cds” | |
| 186 | 1675_at | RAS p21 protein activator (GTPase activating protein) 1 | 0.01 |
| 187 | 1519_at | v-ets avian erythroblastosis virus E26 oncogene homolog 2 | 0.01 |
| 188 | 32172_at | SMART/HDAC1 associated repressor protein | 0.01 |
| 189 | 37480_at | “thrombopoietin (myeloproliferative leukemia virus oncogene ligand, megakaryocyte growth | 0.01 |
| | | and development factor)” | |
| 190 | 39940_at | Cluster Incl. AL080094: Homo sapiens mRNA; cDNA DKFZp564O1262 (from clone | 0.01 |
| | | DKFZp564O1262)/cds = UNKNOWN/gb = AL080094/gi = 5262515/ug = Hs.41185/len = 1062 | |
| 191 | 39068_at | “protein phosphatase 2, regulatory subunit B (B56), delta isoform” | 0.01 |
| 192 | 149_at | “nuclear RNA helicase, DECD variant of DEAD box family” | 0.01 |
| 193 | 36597_at | nucleolar and coiled-body phosphprotein 1 | 0.01 |
| 194 | 37999_at | “coproporphyrinogen oxidase (coproporphyria, harderoporphyria)” | 0.01 |
| 195 | 35114_at | “nuclear receptor subfamily 1, group I, member 2” | 0.01 |
| 196 | 34767_at | modulator of apoptosis 1 | 0.01 |
| 197 | 1403_s_at | small inducible cytokine A5 (RANTES) | 0.01 |
| 198 | 38899_s_at | mitofusin 1 | 0.01 |
| 199 | 41362_at | “ATP-binding cassette, sub-family G (WHITE), member 1” | 0.01 |
| 200 | 33003_at | NCK adaptor protein 2 | 0.01 |
| 201 | 31687_f_at | “hemoglobin, beta” | 0.01 |
| 202 | 38406_f_at | “prostaglandin D2 synthase (21 kD, brain)” | 0.01 |
| 203 | 31623_f_at | metallothionein 1A (functional) | 0.01 |
| 204 | 36996_at | amplified in osteosarcoma | 0.01 |
| 205 | 895_at | macrophage migration inhibitory factor (glycosylation-inhibiting factor) | 0.01 |
| 206 | 38095_i_at | “major histocompatibility complex, class II, DP beta 1” | 0.01 |
| 207 | 38558_at | myelin associated glycoprotein | 0.01 |
| 208 | 31956_f_at | “ribosomal protein, large, P1” | 0.01 |
| 209 | 40448_at | zinc finger protein homologous to Zfp-36 in mouse | 0.01 |
| 210 | 39120_at | metallothionein 1L | 0.01 |
| 211 | 38350_f_at | “tubulin, alpha 2” | 0.01 |
| 212 | 1424_s_at | “D78577/FEATURE = expanded_cds/DEFINITION = D78576S2 Human DNA for 14-3-3 | 0.01 |
| | | protein eta chain, exon2 and complete cds” | |
| 213 | 36681_at | apolipoprotein D | 0.01 |
| 214 | 40886_at | “Cluster Incl. L41498: Homo sapiens longation factor 1-alpha 1 (PTI-1) mRNA, complete cds/ | 0.01 |
| | | cds = (620,1816)/gb = L41498/gi = 927066/ug = Hs.181165/len = 2106” | |
| 215 | 39331_at | “tubulin, beta polypeptide” | 0.01 |
| 216 | 36984_f_at | haptoglobin-related protein | 0.01 |
| 217 | 41288_at | matrix GIa protein | 0.01 |
| 218 | 38637_at | lysyl oxidase | 0.01 |
| 219 | 35278_at | “Cluster Incl. AI541542: libtest16.A02.r Homo sapiens cDNA, 5 end/clone_end = 5/ | 0.01 |
| | | gb = AI541542/gi = 4458915/ug = Hs.539/len = 639” | |
| 220 | 33458_r_at | “H2B histone family, member L” | 0.01 |
| 221 | 32818_at | “hexabrachion (tenascin C, cytotactin)” | 0.01 |
| 222 | 39830_at | ribosomal protein L27 | 0.01 |
| 223 | 34885_at | synaptogyrin 2 | 0.01 |
| 224 | 36152_at | GDP dissociation inhibitor 1 | 0.01 |
| 225 | 41143_at | “Cluster Incl. U12022: Human calmodulin (CALM1) gene/cds = (199,648)/gb = U12022/ | 0.01 |
| | | gi = 2182171/ug = Hs.177656/len = 1526” | |
| 226 | 40580_r_at | parathymosin | 0.01 |
| 227 | 33322_i_at | stratifin | 0.01 |
| 228 | 41753_at | “actinin, alpha 4” | 0.01 |
| 229 | 38972_at | Cluster Incl. AF052169: Homo sapiens clone 24775 mRNA sequence/cds = UNKNOWN/ | 0.01 |
| | | gb = AF052169/gi = 3360480/ug = Hs.109438/len = 1385 | |
| 230 | 41164_at | immunoglobulin heavy constant mu | 0.01 |
| 231 | 35367_at | “lectin, galactoside-binding, soluble, 3 (galectin 3)” | 0.01 |
| 232 | 32612_at | “gelsolin (amyloidosis, Finnish type)” | 0.01 |
| 233 | 40096_at | “ATP synthase, H+ transporting, mitochondrial F1 complex, alpha subunit, isoform 1, cardiac | 0.01 |
| | | muscle” | |
| 234 | 1916_s_at | v-fos FBJ murine osteosarcoma viral oncogene homolog | 0.01 |
| 235 | 38379_at | glycoprotein (transmembrane) nmb | 0.01 |
| 236 | 36736_f_at | phosphoserine phosphatase | 0.01 |
| 237 | 40475_at | calpain 6 | 0.01 |
| 238 | 35837_at | scrapie responsive protein 1 | 0.01 |
| 239 | 34819_at | “CD164 antigen, sialomucin” | 0.01 |
| 240 | 39072_at | MAX-interacting protein 1 | 0.01 |
| 241 | 35965_at | heat shock 70 kD protein 6 (HSP70B‘) | 0.01 |
| 242 | 726_f_at | growth hormone 1 | 0.01 |
| 243 | 32786_at | jun B proto-oncogene | 0.01 |
| 244 | 39741_at | “hydroxyacyl-Coenzyme A dehydrogenase/3-ketoacyl-Coenzyme A thiolase/enoyl-Coenzyme A | |
| | | hydratase (trifunctional protein), beta subunit” | 0.01 |
| 245 | 38750_at | Notch (Drosophila) homolog 3 | 0.01 |
| 246 | 654_at | MAX-interacting protein 1 | 0.01 |
| 247 | 35310_at | Cluster Incl. D45288: HUMHG2121 Homo sapiens cDNA/gb = D45288/gi = 1136684/ | 0.01 |
| | | ug = Hs.57079/len = 1479 | |
| 248 | 32218_at | Cluster Incl. AF034176: AF034176 Homo sapiens cDNA/clone = ntcon5-contig/gb = AF034176/ | 0.01 |
| | | gi = 2707738/ug = Hs.188882/len = 7232 | |
| 249 | 836_at | patched (Drosophila) homolog | 0.01 |
| 250 | 36181_at | LIM and SH3 protein 1 | 0.01 |
| 251 | 38738_at | “SMT3 (suppressor of mif two 3, yeast) homolog 1” | 0.01 |
| 252 | 41634_at | KIAA0256 gene product | 0.01 |
| 253 | 39046_at | histone H2A.F/Z variant | 0.01 |
| 254 | 31740_s_at | paired box gene 4 | 0.01 |
| 255 | 40369_f_at | “Cluster Incl. AL022723: dJ377H14.1 (major histocompatibility complex, class I, G (HLA 6.0))/ | 0.01 |
| | | cds = (120,1127)/gb = AL022723/gi = 5002624/ug = Hs.73885/len = 1508” | |
| 256 | 35298_at | “eukaryotic translation initiation factor 3, subunit 7 (zeta, 66/67 kD)” | 0.01 |
| 257 | 605_at | membrane protein of cholinergic synaptic vesicles | 0.01 |
| 258 | 39704_s_at | high-mobility group (nonhistone chromosomal) protein isoforms I and Y | 0.01 |
| 259 | 117_at | heat shock 70 kD protein 6 (HSP70B‘) | 0.01 |
| 260 | 31873_at | renin-binding protein | 0.01 |
| 261 | 38791_at | dolichyl-diphosphooligosaccharide-protein glycosyltransferase | 0.01 |
| 262 | 37769_at | “endothelial differentiation, lysophosphatidic acid G-protein-coupled receptor, 4” | 0.01 |
| 263 | 39020_at | CD27-binding (Siva) protein | 0.01 |
| 264 | 38378_at | CD53 antigen | 0.01 |
| 265 | 40189_at | SET translocation (myeloid leukemia-associated) | 0.01 |
| 266 | 40437_at | DKFZP564G2022 protein | 0.01 |
| 267 | 38028_at | neuronal specific transcription factor DAT1 | 0.01 |
| 268 | 36791_g_at | tropomyosin 1 (alpha) | 0.01 |
| 269 | 37034_at | putative human HLA class II associated protein I | 0.01 |
| 270 | 35836_at | nuclear distribution gene C (A. nidulans) homolog | 0.01 |
| 271 | 41177_at | hypothetical protein FLJ12443 | 0.01 |
| 272 | 35292_at | HLA-B associated transcript 1 | 0.01 |
| 273 | 1735_g_at | “M60556/FEATURE = mRNA#1/DEFINITION = HUMTGFB3B Human transforming growth | 0.01 |
| | | factor beta-3 gene, 5 end” | |
| 274 | 1100_at | interleukin-1 receptor-associated kinase 1 | 0.01 |
| 275 | 255_s_at | “inhibin, alpha” | 0.01 |
| 276 | 37967_at | lymphocyte antigen 117 | 0.01 |
| 277 | 38855_s_at | neuroblastoma (nerve tissue) protein | 0.01 |
| 278 | 40834_at | KIAA0300 protein | 0.01 |
| 279 | 33332_at | CGI-96 protein | 0.01 |
| 280 | 32815_at | “Cluster Incl. AI687419: tp95h03.x1 Homo sapiens cDNA, 3 end/clone = IMAGE-2207093/ | 0.01 |
| | | clone_end = 3/gb = AI687419/gi = 4898713/ug = Hs.203410/len = 286” | |
| 281 | 35780_at | KIAA0657 protein | 0.01 |
| 282 | 36453_at | KIAA0711 gene product | 0.01 |
| 283 | 37346_at | ADP-ribosylation factor 5 | 0.01 |
| 284 | 31440_at | “transcription factor 7 (T-cell specific, HMG-box)” | 0.01 |
| 285 | 36669_at | FBJ murine osteosarcoma viral oncogene homolog B | 0.01 |
| 286 | 35309_at | “suppression of tumorigenicity 14 (colon carcinoma, matriptase, epithin)” | 0.01 |
| 287 | 34789_at | “serine (or cysteine) proteinase inhibitor, clade B (ovalbumin), member 6” | 0.01 |
| 288 | 555_at | GTP-binding protein homologous to Saccharomyces cerevisiae SEC4 | 0.01 |
| 289 | 36711_at | chromosome 22 open reading frame 5 | 0.01 |
| 290 | 171_at | von Hippel-Lindau binding protein 1 | 0.01 |
| 291 | 41000_at | checkpoint suppressor 1 | 0.01 |
| 292 | 39339_at | KIAA0792 gene product | 0.01 |
| 293 | 40082_at | “fatty-acid-Coenzyme A ligase, long-chain 2” | 0.01 |
| 294 | 36267_at | nuclear factor of kappa light polypeptide gene enhancer in B-cells inhibitor-like 1 | 0.01 |
| 295 | 33683_at | Cluster Incl. D50525: Human mRNA for TI-227H/cds = UNKNOWN/gb = D50525/gi = 1167502/ | 0.01 |
| | | ug = Hs.184914/len = 3911 | |
| 296 | 40044_at | ELL gene (11-19 lysine-rich leukemia gene) | 0.01 |
| 297 | 34060_g_at | “pvt-1 (murine) oncogene homolog, MYC activator” | 0.01 |
| 298 | 33282_at | ladinin 1 | 0.01 |
| 299 | 37279_at | GTP-binding protein overexpressed in skeletal muscle | 0.01 |
| 300 | 38031_at | KIAA0111 gene product | 0.01 |
| 301 | 38011_at | RPB5-mediating protein | 0.01 |
| 302 | 40910_at | “capping protein (actin filament) muscle Z-line, alpha 1” | 0.01 |
| 303 | 1801_at | BRCA1 associated RING domain 1 | 0.01 |
| 304 | 41774_at | ADP-ribosylation factor 4-like | 0.01 |
| 305 | 641_at | presenilin 1 (Alzheimer disease 3) | 0.01 |
| 306 | 39828_at | ADP-ribosylation factor-like 7 | 0.01 |
| 307 | 37147_at | stem cell growth factor; lymphocyte secreted C-type lectin | 0.01 |
| 308 | 36827_at | golgi phosphoprotein 1 | 0.01 |
| 309 | 932_i_at | “zinc finger protein 91 (HPF7, HTF10)” | 0.01 |
| 310 | 1944_f_at | “AF001359/FEATURE=/DEFINITION = AF001359 Homo sapiens DNA mismatch repair | 0.01 |
| | | protein (hMLH1) mRNA, alternatively spliced, partial cds” | |
| 311 | 36208_at | bromodomain-containing 2 | 0.01 |
| 312 | 37217_at | “hemoglobin, zeta” | 0.01 |
| 313 | 39064_at | “5,10-methenyltetrahydrofolate synthetase (5-formyltetrahydrofolate cyclo-ligase)” | 0.01 |
| 314 | 32510_at | “aldo-keto reductase family 7, member A2 (aflatoxin aldehyde reductase)” | 0.01 |
| 315 | 1062_g_at | “interleukin 10 receptor, alpha” | 0.01 |
| 316 | 37679_at | interferon-related developmental regulator 1 | 0.01 |
| 317 | 34080_at | N-acetylated alpha-linked acidic dipeptidase-like; ILEAL DIPEPTIDYLPEPTIDASE | 0.01 |
| 318 | 1477_s_at | “cytochrome P450, subfamily IIC (mephenytoin 4-hydroxylase), polypeptide 18” | 0.01 |
| 319 | 1389_at | “membrane metallo-endopeptidase (neutral endopeptidase, enkephalinase, CALLA, CD10)” | 0.01 |
| 320 | 35781_g_at | KIAA0657 protein | 0.01 |
| 321 | 36987_at | lamin B2 | 0.01 |
| 322 | 36946_at | dual-specificity tyrosine-(Y)-phosphorylation regulated kinase 1A | 0.01 |
| 323 | 36150_at | KIAA0842 protein | 0.01 |
| 324 | 40507_at | “solute carrier family 2 (facilitated glucose transporter), member 1” | 0.01 |
| 325 | 34079_at | inactivation escape 1 | 0.01 |
| 326 | 34808_at | KIAA0999 protein | 0.01 |
| 327 | 37873_g_at | jerky (mouse) homolog | 0.01 |
| 328 | 34799_at | hypothetical protein 24636 | 0.01 |
| 329 | 35615_at | block of proliferation 1 | 0.01 |
| 330 | 34442_at | Cluster Incl. U72943: U72943 Homo sapiens cDNA/gb = U72943/gi = 5763294/ug = Hs.106642/ | 0.01 |
| | | len = 1667 | |
| 331 | 32433_at | ribosomal protein L15 | 0.01 |
| 332 | 40503_at | “myosin-binding protein C, slow-type” | 0.01 |
| 333 | 40014_at | semaphorin Y | 0.01 |
| 334 | 38633_at | metastasis associated 1 | 0.01 |
| 335 | 36807_at | TED protein | 0.01 |
| 336 | 40227_at | “Cluster Incl. D29810: Human mRNA for unknown product, partial cds/cds = (0.1096)/ | 0.01 |
| | | gb = D29810/gi = 704440/ug = Hs.153445/len = 1388” | |
| 337 | 32841_at | zinc finger protein 9 (a cellular retroviral nucleic acid binding protein) | 0.01 |
| 338 | 1850_at | “mutL (E. coli) homolog 1 (colon cancer, nonpolyposis type 2)” | 0.01 |
| 339 | 35956_s_at | pregnancy specific beta-1-glycoprotein 7 | 0.01 |
| 340 | 34287_at | chromosome 21 open reading frame 80 | 0.01 |
| 341 | 35999_r_at | KIAA0781 protein | 0.01 |
| 342 | 1230_g_at | cisplatin resistance associated | 0.01 |
| 343 | 31388_at | early lymphoid activation protein | 0.01 |
| 344 | 41034_s_at | “sulfotransferase family, cytosolic, 2B, member 1” | 0.01 |
| 345 | 32037_r_at | ribonuclease P (14 kD) | 0.01 |
| 346 | 32773_at | “major histocompatibility complex, class II, DQ alpha 1” | 0.01 |
| 347 | 33263_at | Cluster Incl. X67098: H. sapiens rTS alpha mRNA containing four open reading frames/ | 0.01 |
| | | cds = UNKNOWN/gb = X67098/gi = 475908/ug = Hs.180433/len = 1817 | |
| 348 | 40143_at | KIAA0140 gene product | 0.01 |
| 349 | 37509_at | cytokine receptor-like molecule 9 | 0.01 |
| 350 | 39164_at | ariadne (Drosophila) homolog 2 | 0.01 |
| 351 | 39863_at | KIAA0296 gene product | 0.01 |
| 352 | 36214_at | Kruppel-like factor 4 (gut) | 0.01 |
| 353 | 36466_at | “dystrobrevin, alpha” | 0.01 |
| 354 | 38319_at | “CD3D antigen, delta polypeptide (TiT3 complex)” | 0.01 |
| 355 | 38675_at | small nuclear ribonucleoprotein polypeptide C | 0.01 |
| 356 | 39112_at | “upstream transcription factor 2, c-fos interacting” | 0.01 |
| 357 | 38968_at | SH3-domain binding protein 5 (BTK-associated) | 0.01 |
| 358 | 34306_at | muscleblind (Drosophila)-like | 0.01 |
| 359 | 37868_s_at | myelin oligodendrocyte glycoprotein | 0.01 |
| 360 | 36457_at | guanine monphosphate synthetase | 0.01 |
| 361 | 41514_s_at | mitochondrial ribosomal protein L9 | 0.01 |
| 362 | 36313_at | “Cluster Incl. M55267: Human EV12 protein gene/cds = (0.698)/gb = M55267/gi = 182279/ | 0.01 |
| | | ug = Hs.41846/len = 699” | |
| 363 | 1055_g_at | replication factor C (activator 1) 4 (37 kD) | 0.01 |
| 364 | 34629_at | p53-induced protein | 0.01 |
| 365 | 41565_at | ataxin 2 related protein | 0.01 |
| 366 | 36740_at | “double C2-like domains, alpha” | 0.01 |
| 367 | 36580_at | hypothetical protein FLJ13910 | 0.01 |
| 368 | 1680_at | growth factor receptor-bound protein 7 | 0.01 |
| 369 | 35238_at | TNF receptor-associated factor 5 | 0.01 |
| 370 | 733_at | Mucin | 0.01 |
| 371 | 38449_at | hypthetical protein PRO2389 | 0.01 |
| 372 | 41547_at | “BUB3 (budding uninhibited by benzimidazoles 3, yeast) homolog” | 0.01 |
| 373 | 31778_at | “gap junction protein, alpha 8, 50 kD (connexin 50)” | 0.01 |
| 374 | 39255_at | protein C (inactivator of coagulation factors Va and VIIIa) | 0.01 |
| 375 | 33921_at | “glucose-6-phosphatase, transport (glucose-6-phosphate) protein 1” | 0.01 |
| 376 | 41174_at | RAN binding protein 2-like 1 | 0.01 |
| 377 | 33147_at | likely ortholog of mouse zinc finger protein Zfr | 0.01 |
| 378 | 843_at | “protein tyrosine phosphatase type IVA, member 1” | 0.01 |
| 379 | 34069_s_at | “Cluster Incl. S79325: SYT . . . SSX1 {translocation breakpoint} [human, synovial sarcomas, | 0.01 |
| | | mRNA Partial Mutant, 3 genes, 585 nt]/cds = (240,476)/gb = S79325/gi = 1087047/ | |
| | | ug = Hs.194759/len = 585” | |
| 380 | 35317_at | meningioma expressed antigen 5 (hyaluronidase) | 0.01 |
| 381 | 40155_at | actin binding LIM protein 1 | 0.01 |
| 382 | 35763_at | KIAA0540 protein | 0.01 |
| 383 | 41107_at | syntaphilin | 0.01 |
| 384 | 32894_at | leucine-rich neuronal protein | 0.01 |
| 385 | 40788_at | adenylate kinase 2 | 0.01 |
| 386 | 34009_at | cancer/testis antigen 2 | 0.01 |
| 387 | 36079_at | quinone oxidoreductase homolog | 0.01 |
| 388 | 41114_at | KIAA0807 protein | 0.01 |
| 389 | 31731_at | chromobox homolog 4 (Drosophila Pc class) | 0.01 |
| 390 | 32897_at | “5,10-methylenetetrahydrofolate reductase (NADPH)” | 0.01 |
| 391 | 34949_at | KIAA1048 protein | 0.01 |
| 392 | 37506_at | Huntingtin-interacting protein A | 0.01 |
| 393 | 37791_at | “Cluster Incl. N29966: yw53g02.s1 Homo sapiens cDNA, 3 end/clone = IMAGE-255986/ | 0.01 |
| | | clone_end = 3/gb = N29966/gi = 1148486/ug = Hs.125231/len = 496” | |
| 394 | 36964_at | “membrane-bound transcription factor protease, site 1” | 0.01 |
| 395 | 39585_at | cell cycle related kinase | 0.01 |
| 396 | 34845_at | CGI-51 protein | 0.01 |
| 397 | 34953_i_at | “phosphodiesterase 5A, cGMP-specific” | 0.01 |
| 398 | 32246_g_at | putative methyltransferase | 0.01 |
| 399 | 38361_g_at | RAS guanyl releasing protein 2 (calcium and DAG-regulated) | 0.01 |
| 400 | 31794_at | “5′-nucleotidase (purine), cytosolic type B” | 0.01 |
| 401 | 40576_f_at | heterogeneous nuclear ribonucleoprotein D-like | 0.01 |
| 402 | 31579_at | “Cluster Incl. AF005082: Homo sapiens skin-specific protein (xp33) mRNA, partial cds/ | 0.01 |
| | | cds = (0.287)/gb = AF005082/gi = 2589191/ug = Hs.113261/len = 303” | |
| 403 | 31314_at | bone morphogenetic protein 3 (osteogenic) | 0.01 |
| 404 | 40197_at | HYA22 protein | 0.01 |
| 405 | 38394_at | KIAA0089 protein | 0.01 |
| 406 | 36770_at | “signal transducer and activator of transcription 2, 113 kD” | 0.01 |
| 407 | 39976_at | KIAA1785 protein | 0.01 |
| 408 | 1281_f_at | Serine/Threonine Kinase | 0.01 |
| 409 | 36553_at | acetylserotonin O-methyltransferase-like | 0.01 |
| 410 | 31892_at | “protein tyrosine phosphatase, receptor type, M” | 0.01 |
| 411 | 32067_at | cAMP responsive element modulator | 0.01 |
| 412 | 35727_at | hypothetical protein FLJ20517 | 0.01 |
| 413 | 35524_at | “complement component 8, gamma polypeptide” | 0.01 |
| 414 | 33392_at | DKFZP434J154 protein | 0.01 |
| 415 | 36531_r_at | hypothetical protein | 0.01 |
| 416 | 32978_g_at | chromosome 6 open reading frame 32 | 0.01 |
| 417 | 37907_at | coagulation factor VIII-associated (intronic transcript) | 0.01 |
| 418 | 34585_at | distal-less homeo box 2 | 0.01 |
| 419 | 32845_at | heparan sulfate proteoglycan 2 (perlecan) | 0.01 |
| 420 | 37966_at | “parvin, beta” | 0.01 |
| 421 | 37188_at | phosphoenolpyruvate carboxykinase 2 (mitochondrial) | 0.01 |
| 422 | 478_g_at | interferon regulatory factor 5 | 0.01 |
| 423 | 32647_at | vesicle-associated soluble NSF attachment protein receptor (v-SNARE; homolog of S. | 0.01 |
| | | cerevisiae VTI1) | |
| 424 | 35540_at | hyaluronoglucosaminidase 3 | 0.01 |
| 425 | 37601_at | “solute carrier family 22 (extraneuronal monoamine transporter), member 3” | 0.01 |
| 426 | 32124_at | hypothetical protein LOC57187 | 0.01 |
| 427 | 36642_at | “Cluster Incl. J00287: Human pepsinogen gene/cds = (55,1221)/gb = J00287/gi = 189798/ | 0.01 |
| | | ug = Hs.75558/len = 1381” | |
| 428 | 35989_at | calcineurin-binding protein calsarcin-1 | 0.01 |
| 429 | 349_g_at | kinesin-like 2 | 0.01 |
| 430 | 33173_g_at | hypothetical protein FLJ10849 | 0.01 |
| 431 | 31539_r_at | “Cluster Incl. L23852: Homo sapiens (clone Z146) retinal mRNA, 3 end and repeat region/ | 0.01 |
| | | cds = (0.241)/gb = L23852/gi = 393126/ug = Hs.73838/len = 1711” | |
| 432 | 34816_at | trinucleotide repeat containing 12 | 0.01 |
| 433 | 32856_at | KIAA0819 protein | 0.01 |
| 434 | 31662_at | vacuolar protein sorting 45A (yeast homolog) | 0.01 |
| 435 | 40516_at | aryl hydrocarbon receptor | 0.01 |
| 436 | 35622_at | neuronal Shc adaptor homolog | 0.01 |
| 437 | 36653_g_at | uroporphyrinogen III synthase (congenital erythropoietic porphyria) | 0.01 |
| 438 | 1150_at | “protein tyrosine phosphatase, receptor type, E” | 0.01 |
| 439 | 1229_at | cisplatin resistance associated | 0.01 |
| 440 | 36625_at | peroxisomal long-chain acyl-coA thioesterase | 0.01 |
| 441 | 40572_at | “Cluster Incl. N51314: yz15b04.s1 Homo sapiens cDNA, 3 end/clone = IMAGE-283087/ | 0.01 |
| | | clone_end = 3/gb = N51314/gi = 1192480/ug = Hs.170241/len = 472” | |
| 442 | 31897_at | downregulated in ovarian cancer 1 | 0.01 |
| 443 | 558_at | keratin 1 (epidermolytic hyperkeratosis) | 0.01 |
| 444 | 39751_at | DHHC1 protein | 0.01 |
| 445 | 40892_s_at | DNA segment on chromosome X (unique) 9879 expressed sequence | 0.01 |
| 446 | 526_s_at | postmeiotic segregation increased (S. cerevisiae) 2 | 0.01 |
| 447 | 359_at | “interleukin 13 receptor, alpha 1” | 0.01 |
| 448 | 36456_at | DKFZP564I052 protein | 0.01 |
| 449 | 39298_at | “alpha2,3-sialyltransferase” | 0.01 |
| 450 | 1495_at | latent transforming growth factor beta binding protein 1 | 0.01 |
| 451 | 31649_at | HGC6.1.1 protein | 0.01 |
| 452 | 41722_at | nicotinamide nucleotide transhydrogenase | 0.01 |
| 453 | 38204_at | KIAA0406 gene product | 0.01 |
| 454 | 1885_at | “excision repair cross-complementing rodent repair deficiency, complementation group 3 | 0.01 |
| | | (xeroderma pigmentosum group B complementing)” | |
| 455 | 36005_at | suppressor of white apricot homolog 2 | 0.01 |
| 456 | 37180_at | “phospholipase C, gamma 2 (phosphatidylinositol-specific)” | 0.01 |
| 457 | 33354_at | E3 ubiquitin ligase SMURF2 | 0.01 |
| 458 | 32624_at | DKFZP566D133 protein | 0.01 |
| 459 | 32132_at | KIAA0675 gene product | 0.01 |
| 460 | 33411_g_at | “integrin, alpha 6” | 0.01 |
| 461 | 34675_at | Cluster Incl. AL080210: Homo sapiens mRNA; cDNA DKFZp586G0623 (from clone | 0.01 |
| | | DKFZp586G0623)/cds = UNKNOWN/gb = AL080210/gi = 5262699/ug = Hs.23437/len = 1388 | |
| 462 | 37710_at | “MADS box transcription enhancer factor 2, polypeptide C (myocyte enhancer factor 2C)” | 0.01 |
| 463 | 794_at | “protein tyrosine phosphatase, non-receptor type 6” | 0.01 |
| 464 | 39016_r_at | keratin 6A | 0.01 |
| 465 | 1909_at | B-cell CLL/lymphoma 2 | 0.01 |
| 466 | 36564_at | Cluster Incl. W27419: 31a10 Homo sapiens cDNA/gb = W27419/gi = 1307241/ug = Hs.64239/ | 0.01 |
| | | len = 803 | |
| 467 | 622_at | “RAB6A, member RAS oncogene family” | 0.01 |
| 468 | 40201_at | dopa decarboxylase (aromatic L-amino acid decarboxylase) | 0.01 |
| 469 | 31894_at | centromere protein C 1 | 0.01 |
| 470 | 41100_at | tumor up-regulated CARD-containing antagonist of caspase nine | 0.01 |
| 471 | 33202_f_at | Friedreich ataxia | 0.01 |
| 472 | 394_at | bleomycin hydrolase | 0.01 |
| 473 | 31485_at | “Cluster Incl. M57423: Homo sapiens phosphoribosylpyrophosphate synthetase subunit III | 0.01 |
| | | mRNA, 3 end/cds = (81,1037)/gb = M57423/gi = 190521/ug = Hs.169284/len = 1091” | |
| 474 | 32962_at | cystathionase (cystathionine gamma-lyase) | 0.01 |
| 475 | 39480_s_at | KIAA1454 protein | 0.01 |
| 476 | 362_at | “protein kinase C, zeta” | 0.01 |
| 477 | 33270_i_at | Dmx-like 1 | 0.01 |
| 478 | 40086_at | KIAA0261 protein | 0.01 |
| 479 | 966_at | RAD54 (S.cerevisiae)-like | 0.01 |
| 480 | 1108_s_at | EphA1 | 0.01 |
| 481 | 962_at | BMX non-receptor tyrosine kinase | 0.01 |
| 482 | 1610_s_at | “J00139/FEATURE = cds/DEFINITION = HUMFOL5 Human dihydrofolate reductase gene, | 0.01 |
| | | exon 6 and 3 flank” | |
| 483 | 35650_at | KIAA0356 gene product | 0.01 |
| 484 | 35025_at | “small inducible cytokine subfamily B (Cys-X-Cys), member 5 (epithelial-derived neutrophil- | 0.01 |
| | | activating peptide 78)” | |
| 485 | 1529_at | hypothetical protein CG003 | 0.01 |
| 486 | 177_at | “phospholipase D1, phophatidylcholine-specific” | 0.01 |
| 487 | 496_s_at | “interleukin 11 receptor, alpha” | 0.01 |
| 488 | 33998_at | neurotensin | 0.01 |
| 489 | 1384_at | “M64930/FEATURE=/DEFINITION = HUMPROP2AB Human protein phosphatase 2A | 0.01 |
| | | beta subunit mRNA, complete cds” | |
| 490 | 36898_r_at | “primase, polypeptide 2A (58 kD)” | 0.01 |

What is claimed is:

1. A method of profiling a tumor/cancer in human tissue specimens, comprising:
(a) exposing said human tissue specimens to one or a plurality of reagents to one or a plurality of products of genes;
(b) measuring quantitatively the levels of said one or said plurality of products of genes in said tissue specimens; and
(c) profiling said tumor/cancer from the quantitative levels of the said products of genes from step 1 (b).

2. The method of claim 1, wherein said human tissue specimens is selected from a group consisting human tissue extracts, human cells, human tissues, organs, blood, blood serum, body fluids and a combination thereof

3. The method of claim 1, wherein said human tissue specimens is blood serum.

4. The method of claim 1, wherein said tumor/cancer is a prostate cancer.

5. The method of claim 1, wherein said tumor/cancer is a glioblastoma.

6. The method of claim 1, wherein said tumor/cancer is a breast cancer.

7. The method of claim 1, wherein said gene is selected from a group consisting listed genes in the TABLE 2 of the specifications of this application.

8. The method of claim 1, wherein said genes comprises listed genes in the TABLE 2 of the specifications of this application.

9. The method of claim 1, wherein said gene is selected from a group consisting insulin-like growth factor binding protein 2 or IGFBP2 (GenBank Accession numbers of X16302 and S37730), a hypothetical protein (GenBank Accession number of AF052186), TUA8 Cri-du-chat region (GenBank Accession number of AF009314), dual specificity phosphatase 10 or MPK-5 (GenBank Accession number of AB026436), Neuralized (GenBank Accession number of AF029729), regulator of G-protein signaling 1 or RGS-1 (GenBank Accession number of S59049), expressed in activated T/LAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTK1 (GenBank Accession number of AB02064 1), and pleckstrin homology, sec 7 and coiled/coid domains 1 or cytohesin 1 (GenBank Accession number ofM85169) and a combination thereof.

10. The method of claim 1, wherein said genes comprises insulin-like growth factor binding protein 2 or IGFBP2 (GenBank Accession numbers of X16302 and S37730), a hypothetical protein (GenBank Accession number ofAF052186), TUA8 Cri-du-chat region (GenBank Accession number of AF009314), dual specificity phosphatase 10 or MPK-5 (GenBank Accession number of AB026436), Neuralized (GenBank Accession number of AF029729), regulator of G-protein signaling 1 or RGS-I (GenBank Accession number of S59049), expressed in activated T/LAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTKI (GenBank Accession number of AB020641), and pleckstrin homology, sec 7 and coiled/coid domains 1 or cytohesin 1 (GenBank Accession number of M85169).

11. The method of claim 1, wherein said gene is insulin-like growth factor binding protein 2 or IGFBP2 (GenBank Accession numbers of X16302 and S37730).

12. The method of claim 1, wherein said products of genes is selected from the group consisting of gene mRNA transcripts, proteins encoded by genes, modifications of the encoded proteins and a combination thereof.

13. The method of claim 1, wherein said reagents is selected from a group consisting monoclonal antibody, polyclonal antibody, nucleic acid of either RNA or DNA, polynucleotide, aptamer, other binders to a protein and a combination thereof.

14. The method of claim 1, wherein said reagent is an antibody against insulin-like growth factor binding protein 2 or IGFBP2 (GenBank Accession numbers of X16302 and S37730).

15. The method of claim 1, wherein said measuring is performed using methods selected from a group consisting of molecular hybridization methods such as Northern blot, In situ hybridization, branched DNA methods, rolling cycle amplication (RCA), RNA transcription methods, gene chip methods, cDNA microarray, polymerase chain reaction (PCR), reverse transcription-PCR (RT-PCR), quantitative PCR (Q-PCR), Western blot, immunocytochemistry, immunohistochemistry, fluorescent cell sorting, and a combination thereof.

16. The method of claim 1, wherein said profiling is assessing, diagnosis or prognosis of PTEN tumor suppressor gene abnormality status such as PTEN tumor suppressor gene mutations, deletions, aberrant or absent PTEN mRNA or PTEN protein.

17. The method of claim 1, wherein said profiling is assessing, diagnosis or prognosis of PTEN-related signal transduction pathway and its responsiveness to said pathway modulators such as agonists or antagonists.

18. The method of claim 17, wherein said PTEN-related signal transduction pathway is the PI3K-Akt pathways.

19. The method of claim 17, wherein said modulator is an antagonist or inhibitor.

20. The method of claim 19, wherein said antagonist is an Akt inhibitors.

21. A method of screening a compound inhibits cancer cell growth, comprising:
(a) exposing said cancer cells treated with and without said compound to one or a plurality of reagents to one or a plurality of products of genes;
(b) measuring quantitatively the levels of upregulation or down-regulation of said one or said plurality of products of said genes in said compound-treated vs. untreated cancer cells; and
(c) assessing said cancer cell responsiveness to the compound treatment from the quantitative levels of the upregulation or down-regulation of said products of said genes from step 21((b).

22. The method of claim 21, wherein said cancer cell is of established cancer cell line or primary cancer cell culture.

23. The method of claim 21, wherein said cancer cell is a prostate cancer cell.

24. The method of claim 21, wherein said cancer cell is a glioblastoma cell.

25. The method of claim 21, wherein said cancer cell is a breast cancer cell.

26. The method of claim 21, wherein said compound is selected from a group consisting small molecule chemical compound, peptide, nucleic acid, oligonucleotide, antibody, aptamer, a modification thereof and a combination thereof.

27. The method of claim 21, wherein said gene is selected from a group consisting listed genes in the TABLE 2 of the specifications of this application.

28. The method of claim 21, wherein said genes comprises listed genes in the TABLE 2 of the specifications of this application.

29. The method of claim 21, wherein said gene is selected from a group consisting insulin-like growth factor binding protein 2 or IGFBP2 (GenBank Accession numbers of XI 6302 and S37730), a hypothetical protein (GenBank Accession number of AF052186), TUA8 Cri-du-chat region (GenBank Accession number of AF009314), dual specificity phosphatase 10 or MPK-5 (GenBank Accession number of AB026436), Neuralized (GenBank Accession number of AF029729), regulator of G-protein signaling 1 or RGS-1 (GenBank Accession number of S59049), expressed in activated T/LAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTK1 (GenBank Accession number of AB020641), and pleckstrin homology, sec 7 and coiled/coid domains 1 or cytohesin 1 (GenBank Accession number of M85169) and a combination thereof.

30. The method of claim 21, wherein said genes comprises insulin-like growth factor binding protein 2 or IGFBP2 (GenBank Accession numbers of X16302 and S37730), a hypothetical protein (GenBank Accession number of AF052186), TUA8 Cri-du-chat region (GenBank Accession number of AF009314), dual specificity phosphatase 10 or MPK-5 (GenBank Accession number of AB026436), Neuralized (GenBank Accession number of AF029729), regulator of G-protein signaling 1 or RGS-1 (GenBank Accession number of S59049), expressed in activated T/LAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTKI (GenBank Accession number of AB020641), and pleckstrin homology, sec 7 and coiled/coid domains 1 or cytohesin 1 (GenBankAccession number of M85169).

31. The method of clairn 21, wherein said gene is insulin-like growth factor binding protein 2 or IGFBP2 (GenBank Accession numbers of X16302 and S37730).

32. The method of claim 21, wherein said products of genes is selected from the group consisting of gene mRNA transcripts, proteins encoded by genes, modifications of the encoded proteins and a combination thereof.

33. The method of claim 21, wherein said reagents is selected from a group consisting monoclonal antibody, polyclonal antibody, nucleic acid of either RNA or DNA, polynucleotide, aptamer, other binders to a protein and a combination thereof.

34. The method of claim 21, wherein said reagent is an antibody against insulin-like growth factor binding protein 2 or IGFBP2 (GenBank Accession numbers of X16302 and S37730).

35. The method of claim 21, wherein said measuring is performed using methods selected from a group consisting of molecular hybridization methods such as Northern blot, in situ hybridization, branched DNA methods, rolling cycle amplication (RCA), RNA transcription methods, gene chip methods, cDNA microarray, polymerase chain reaction (PCR), reverse transcription-PCR (RT-PCR), quantitative PCR (Q-PCR), Western blot, immunocytochemistry, immunohistochemistry, fluorescent cell sorting, and a combmation thereof.

36. The method of claim 21, wherein said compound is targeting PTEN-related signal transduction pathway.

37. The method of claim 36, wherein said PTEN-related signal transduction pathway is the PI3K-Akt pathways.

38. The method of claim 21, wherein said compound is a PI3K-Akt pathway inhibitor.

39. The method of claim 38, wherein said compound is an Akt inhibitor.

40. The method of claim 21, wherein said compound is a modulator of said products of said genes.

41. The method of claim 40, wherein said modulator is either an agonist or an antagonist of said products of said genes.

42. The method of claim 21, wherein said gene is insulin-like growth factor binding protein 2 or IGFBP2 (GenBank Accession numbers of X16302 and S37730).

43. The method of claim 21, wherein said compound is an antibody against said insulin-like growth factor binding protein 2 or IGFBP2 (GenBank Accession numbers of X16302 and S37730).

44. An assay kit of profiling a tumor/cancer in human tissue specimens, comprising one or a plurality of reagents to one or a plurality of products of genes;

45. The assay kit of claim 44, wherein said human tissue specimens is selected from a group consisting human tissue extracts, human cells, human tissues, organs, blood, blood serum, body fluids and a combination thereof.

46. The assay kit of claim 44, wherein said human tissue specimens is blood serum.

47. The assay kit of claim 44, wherein said tumor/cancer is a prostate cancer.

48. The assay kit of claim 44, wherein said tumor/cancer is a glioblastoma.

49. The assay kit of claim 44, wherein said tumor/cancer is a breast cancer.

50. The assay kit of claim 44, wherein said gene is selected from a group consisting listed genes in the TABLE 2 of the specifications of this application.

51. The assay kit of claim 44, wherein said genes comprises listed genes in the TABLE 2 of the specifications of this application.

52. The assay kit of claim 44, wherein said gene is selected from a group consisting insulin-like growth factor binding protein 2 or IGFBP2 (GenBank Accession numbers of X16302 and S37730), a hypothetical protein (GenBank Accession number of AF052186), TUA8 Cri-du-chat region (GenBank Accession number of AF009314), dual specificity phosphatase 10 or MPK-5 (GenBank Accession number of AB026436), Neuralized (GenBank Accession number of AF029729), regulator of G-protein signaling 1 or RGS-1 (GenBank Accession number of S59049), expressed in activated T/LAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTKL (GenBank Accession number of AB020641), and pleckstrin homology, sec 7 and coiled/coid domains 1 or cytohesin 1 (GenBank Accession number of M85169) and a combination thereof.

53. The assay kit of claim 44, wherein said genes comprises insulin-like growth factor binding protein 2 or IGFBP2 (GenBank Accession numbers of XI 6302 and S37730), a hypothetical protein (GenBank Accession number of AF052186), TUA8 Cri-du-chat region (GenBank Accession number ofAF009314), dual specificity phosphatase 10 or MPK-5 (GeiBank Accession number of AB026436), Neuralized (GenBank Accession number of AF029729), regulator of G-protein signaling 1 or RGS-1 (GenBank Accession number of S59049), expressed in activated TALAK lymphcytes or LAP-4p (GenBank Accession number of AB002405), gamma-tubulin complex protein 2 or GCP2 (GenBank Accession number of AF042379), human AMP deaminase gene or AMPD3 (GenBank Accession number of U29926), PFTAIRE protein kinase 1 or PFTK1 (GenBank Accession number of AB020641), and pleckstrin homology, sec 7 and coiled/coid domains 1 or cytohesin 1 (GenBank Accession number of M85169).

54. The assay kit of claim 44, wherein said gene is insulin-like growth factor binding protein 2 or IGFBP2 (GenBank Accession numbers of X16302 and S37730).

55. The assay kit of claim 44, wherein said products of genes is selected from the group consisting of gene mRNA transcripts, proteins encoded by genes, modifications of the encoded proteins and a combination thereof.

56. The assay kit of claim 44, wherein said reagents is selected from a group consisting monoclonal antibody, polyclonal antibody, nucleic acid of either RNA or DNA, polynucleotide, aptamer, other binders to a protein and a combination thereof.

57. The assay kit of claim 44, wherein said reagent is an antibody against insulin-like growth factor binding protein 2 or IGFBP2 (GenBank Accession numbers of XI 6302 and S37730).

58. The assay kit of claim 44, wherein said assay kit is useful for methods selected from a group consisting of molecular hybridization methods such as Northern blot, in situ hybridization, branched DNA methods, rolling cycle amplication (RCA), RNA transcription methods, gene chip methods, cDNA microarray, polymerase chain reaction (PCR), reverse transcription-PCR (RT-PCR), quantitative PCR (Q-PCR), Western blot, immunocytochemistry, immunohistochemistry, fluorescent cell sorting, and a combination thereof.

59. A therapeutic useful antibody against insulin-like growth factor binding protein 2 or IGFBP2 (GenBank Accession numbers of X16302 and S37730).

60. The said antibody of claim 59 is a neutralizing antibody.
